(19) United States
(12) Patent Application Publication
(10) Pub. No.: US 2008/0009420 A1
(43) Pub. Date: Jan. 10, 2008

(54) Isothermal methods for creating clonal single molecule arrays

(76) Inventors: Gary Paul Schroth, Hayward, CA (US); David Harley Lloyd, Belmont, CA (US); Lu Zhang, Hayward, CA (US); Tobias William Barrost, Saffron Walden (GB); Roberto Rigatti, Saffron Walden (GB); Jonathan Mark Boutell, Saffron Walden (GB)

Correspondence Address: KLAUBER & JACKSON, 411 HACKENSACK AVENUE, HACKENSACK, NJ, 07601, US

(21) Appl. No.: 11/725,597

(22) Filed: Mar. 19, 2007

Related U.S. Application Data
(60) Provisional application No. 60/783,618, filed on Mar. 17, 2006.

(51) Int. Cl. C40B 40/06; C12P 19/34; C40B 50/06

(52) U.S. Cl. 506/16; 435/91.2; 506/26

(57) ABSTRACT

The present invention is directed to a method for isothermal amplification of a plurality of different target nucleic acids, wherein the different target nucleic acids are amplified using universal primers and colonies produced thereby can be distinguished from each other. The method, therefore, generates distinct colonies of amplified nucleic acid sequences that can be analyzed by various means to yield information particular to each distinct colony.

### CROSS REFERENCE TO RELATED APPLICATION

This application claims priority under 35 USC §119(e) from U.S. Provisional Application Ser. No. 60/783,618, filed Mar. 17, 2006, which application is herein specifically incorporated by reference in its entirety.

### FIELD OF THE INVENTION

The invention relates to methods for amplifying polynucleotide sequences and in particular relates to isothermal methods for amplification of polynucleotide sequences. The methods according to the present invention are particularly suited to solid phase amplification utilising flow cells.

### BACKGROUND TO THE INVENTION

Several publications and patent documents are referenced in this application in order to more fully describe the state of the art to which this invention pertains. The disclosure of each of these publications and documents is incorporated by reference herein.

The Polymerase Chain Reaction or PCR (Saiki et al 1985, Science 230:1350) has become a standard molecular biology technique which allows for amplification of nucleic acid molecules. This in-vitro method is a powerful tool for the detection and analysis of small quantities of nucleic acids and other recombinant nucleic acid technologies.

Briefly, PCR requires a number of components: a target nucleic acid molecule, a molar excess of a forward and reverse primer which bind to the target nucleic acid molecule, deoxyribonucleoside triphosphates (DATP, dTTP, dCTP and dGTP) and a polymerase enzyme.

The PCR reaction is a DNA synthesis reaction that depends on the extension of the forward and reverse primers annealed to opposite strands of a dsDNA template that has been denatured (melted apart) at high temperature (90° C. to 100° C.). Using repeated melting, annealing and extension steps usually carried out at differing temperatures, copies of the original template DNA are generated.

Although there have been many improvements and modifications to the original PCR procedure, many of these continue to rely on thermocycling of the reaction mixture, whereby melting, annealing and extension are performed at different temperatures. The major disadvantage of thermocycling reactions relates to the long ‘lag’ times during which the temperature of the reaction mixture is increased or decreased to the correct level. These lag times increase considerably the length of time required to perform an amplification reaction. Hence, thermocycling generally requires the use of expensive and specialised equipment.

Moreover, as a result of the high temperatures used during PCR, the reaction mixtures are subject to evaporation. Consequently PCR reactions are carried out in sealed reaction vessels. The use of such sealed reaction vessels has further disadvantages: as amplification progresses, depletion of dNTP's can become limiting, lowering the efficiency of the reaction. Repeated high temperature cycling can also lead to a reduction in the efficiency of the polymerase enzyme; the half life of Taq polymerase may be as low as 40 minutes at 94° C. and 5 minutes at 97° C. (Wu et al. 1991, DNA and Cell Biology 10, 233-238; Landegren U. 1993, Trends Genet 9, 199-204; Saiki et al. 1988, Science, 239, 487-491). Use of a sealed reaction vessel also makes it difficult to alter or add further reaction components.

To overcome these technical disadvantages, a number of methods have been developed which enable isothermal amplification of nucleic acids.

Strand Displacement Amplification (SDA) (Westin et al 2000, Nature Biotechnology, 18, 199-202; Walker et al 1992, Nucleic Acids Research, 20, 7, 1691-1696), for example, is an isothermal, in vitro nucleic acid amplification technique based upon the ability of a restriction endonuclease such as HincII or BsoBI to nick the unmodified strand of a hemiphosphorothioate form of its recognition site, and the ability of an exonuclease deficient DNA polymerase such as Klenow exo minus polymerase, or Bst polymerase, to extend the 3′-end at the nick and displace the downstream DNA strand. Exponential amplification results from coupling sense and antisense reactions in which strands displaced from a sense reaction serve as targets for an antisense reaction and vice versa. In the original design (G. T. Walker, M. C. Little, J. G. Nadeau and D. D. Shank (1992) Proc. Natl. Acad. Sci 89, 392-396), the target DNA sample is first cleaved with a restriction enzyme(s) in order to generate a double-stranded target fragment with defined 5′- and 3′-ends. Heat denaturation of the double stranded target fragment generates two single DNA strand fragments. Two DNA primers which are present in excess and contain a HincII restriction enzyme recognition sequence bind to the 3′ ends of one or other of the two strands. This generates duplexes with overhanging 5′ ends. A 5′-3′ exonuclease deficient DNA polymerase extends the 3′ ends of the duplexes using three unmodified dNTP's and a modified deoxynucleoside 5[alpha thio]triphosphate which thus produces hemiphosphorothioate recognition sites. The restriction endonuclease nicks the unprotected primer strands of the hemiphosphorothioate recognition site leaving intact the modified complementary strands. The DNA polymerase extends the 3′ end nick and displaces the downstream strand. Nicking and polymerisation/displacement steps cycle continuously because extension at the nick regenerates a nickable HincII recognition site.

There are a number of problems associated with this method. Firstly, the restriction step limits the choice of target DNA sequences since the target must be flanked by convenient restriction sites. Also the restriction enzyme site cannot be present in the target DNA sequence, which makes amplification of multiple target DNA sequences impractical. Secondly, the target DNA must typically be double stranded for restriction enzyme cleavage.

With respect to the surface bound SDA reaction described by Westin et al. (supra), additional disadvantages arise from the fact that the amplified strands are displaced into solution. Unless the individual template strands are kept isolated from each other, the strands can diffuse and cause mixing of sequences. Westin et al. control this by using specific amplification primers for each target to be amplified.

For the multiplex analysis of large numbers of target fragments having different sequences, it is desirable to perform a simultaneous amplification reaction of the plurality of targets in a single mixture, using a single pair of primers for amplification of all the targets. Such universal amplification reactions are described more fully in application WO09844151 (Method of Nucleic Acid Amplification). For the amplification of isolated single molecules on a planar surface, it is advantageous to maintain the nucleic acid strands in a surface bound state throughout the entire amplification process so as to prevent cross-contamination of sequences. Methods such as SDA, as reported by Westin et al., do not allow for universal amplification of multiple fragments having different sequences in a combined mixture because the fragments can diffuse freely in solution during the amplification process, thereby necessitating a reliance on individual primers/primer sets that are specific for each fragment to be amplified.

Loop-mediated Isothermal Amplification (LAMP) is a nucleic acid amplification method that amplifies DNA under isothermal conditions (Notomi et al, Nucleic Acids Res 2000; 28:e63).

The LAMP method requires a set of four specially designed primers and a DNA polymerase with strand displacement activity to produce amplification products which are stem-loop DNA structures. The four primers recognise a total of six distinct sequences of the target DNA. An inner primer containing sequences of the sense and antisense strands of the target DNA initiates LAMP. DNA synthesis of a following strand primed by an outer primer displaces a single stranded DNA. This displaced strand serves as a template for DNA synthesis primed by the second inner and outer primers that hybridise to the other end of the target to produce a stem-loop DNA structure. In subsequent steps one inner primer hybridises to the loop on the product and initiates displacement DNA synthesis. This yields the original stem-loop DNA and a new stem-loop DNA with a stem twice the length of the original.

Major disadvantages of this method include the necessity of preparing sets of specially designed primers that must be designed based on known sequences. This makes multiplex reactions of different targets difficult. In addition, since the amplification products are stem-loop DNAs which must be further digested with restriction enzymes, there is the possibility that the target DNA will contain restriction sites and be cleaved.

Isothermal and Chimeric primer-initiated Amplification of Nucleic acids or ICAN is an isothermal DNA amplification method using exo-Bca DNA polymerase, RNaseH and DNA-RNA chimeric primers (Shimada et al, Rinsho Byori 2003, November; 51(11):1061-7). In this method a target nucleic acid is amplified by an enzymatic system similar to SDA. Chimeric primers consisting of a DNA portion and an RNA portion are annealed to a target nucleic acid and extended by polymerase activity. As the primers are displaced, complementary strands are displaced. RNase H nicks the chimeric primer which is then extended with subsequent strand displacement. The disadvantages of this method include the necessity of a DNA:RNA composite primer and the difficulties associated with amplifying more than one target nucleic acid sequence. In addition, copied/amplified products are produced in long linear strands which may require restriction enzyme cleavage prior to further analyses steps, or may be lost from the surface by a single strand breakage event.

Rolling circle amplification (Lizardi et al. 1998, Nature Genetics, 19:225-232) is another method of amplifying single stranded molecules (in this case circles of nucleic acids) that relies on the template strand for amplification remaining in free solution. Amplification of circles of multiple different sequences relies on either multiple anchored primers with template specific sequences, or on the use of circular molecules containing universal primer regions. There are several limitations that restrict the applicability of this method with respect to solid phase amplification. To begin, the circles can diffuse freely in solution, thereby permitting multiple seeding events for each circle, which in turn prevents sequestration of sequences generated. The method suffers from the additional drawback that the very long linear amplicons generated are attached to the surface by a single covalent bond, breakage of which would result in a loss of the entire signal from the surface. It is noteworthy that in a process involving multiple cycles of sequencing over an extended period of hours or days, under multiple flow conditions, and in different temperatures and buffers, the chances of a strand breaking event are quite high. Hence, if the whole signal is only attached via a single point attachment, a strand breaking event could cause the whole sequence read to be lost in the middle of the experiment.

In WO00/41524, the applicants disclose an in vitro method to amplify DNA exponentially at a constant temperature using a DNA polymerase and accessory proteins, but excluding the use of exogenously added primers. This method uses a helicase enzyme to separate the DNA strands and requires binding proteins to prevent the separated strands from re-annealing. Such a method is, however, not efficient since the accessory binding proteins need to be displaced for amplification to occur.

U.S. Pat. No. 6,277,605 discloses a method of isothermal amplification which utilises cycling the concentration of divalent metal ions to denature DNA. This method suffers from a number of disadvantages: the first of these relates to the specialised electrolytic equipment required. The second disadvantage is that at low temperature the specificity of primer binding is low, resulting in the generation of non-specific amplification products.

WO02/46456 describes a method of isothermal amplification of nucleic acids immobilised on a solid support. This method uses mechanical stress and the curvature of a DNA molecule to destabilise and separate at least a part of a DNA duplex to allow primer binding under isothermal conditions.

U.S. Pat. No. 5,939,291 discloses a method of isothermal amplification which uses electrostatic-based denaturation and separation of nucleic acids. The applicants demonstrate a method of nucleic acid amplification which involves attaching and detaching nucleic acids to a solid support. The applicants do not disclose the use of nucleic acids and primers immobilised to the same solid surface nor are the methods presented suitable for isothermal amplification of nucleic acids to form clusters for sequencing by synthesis, as the different target sequences will become intermingled after removal from the surface.

U.S. Pat. No. 6,406,893 discloses a method of isothermal amplification in a microfluidic chamber where the nucleic acid solution is pumped between different reagents to cause denaturing and renaturing. This methodology may be useful for the amplification of tiny amounts of individual target sequences, but is not amenable to multiplexing a variety of samples since the nucleic acids are not immobilised.

### SUMMARY OF THE INVENTION

The present inventors have discovered a method of isothermal amplification of target nucleic acids on a planar surface which allows efficient amplification without the intermingling of different target sequences. Accordingly, the instant method facilitates isothermal amplification of a plurality of different target nucleic acids (i.e., targets comprising different nucleic acid sequences) using universal primers, wherein colonies produced thereby are positionally distinct or isolated from each other. The method, therefore, generates distinct colonies of amplified nucleic acid sequences that can be analyzed by various means to yield information particular to each distinct colony.

In a first aspect, the invention provides a method for isothermally amplifying single stranded nucleic acid molecules immobilized on a planar solid surface comprising:
i) providing a planar solid surface comprising at least one 5′-end immobilized first single stranded nucleic acid template molecule comprising a sequence Y at the 5′ end and a sequence Z at the 3′ end and a plurality of first and second primers comprising sequences X and Y immobilized at their 5′ ends, wherein sequence X is hybridizable to sequence Z;
ii) annealing said at least one 5′-end immobilized first single stranded nucleic acid template molecule to said first immobilized primers, wherein the first sequence Z of each template molecule is annealed to one of said first immobilized primers comprising sequence X;
iii) performing a primer extension reaction using primer annealed 5′-end immobilized first single stranded nucleic acid template molecules to generate double stranded nucleic acid molecules comprising 5′-end immobilized first and second single stranded nucleic acid molecules, wherein the 5′-end immobilized second single stranded nucleic acid molecules are complementary copies of the 5′-end immobilized first single stranded template nucleic acid molecules and each of the 5′-end immobilized second single stranded nucleic acid molecules comprises a sequence at the 3′ end that is hybridizable to the second primer sequence Y;
iv) flowing a chemical denaturant across the planar solid surface to denature said double stranded nucleic acid molecules to generate 5′-end immobilized first and second single stranded nucleic acid molecules;
v) removing the chemical denaturant and annealing said 5′-end immobilized first and second single stranded nucleic acid molecules to said first and second immobilized primers comprising sequences X and Y;
vi) performing a primer extension reaction using primer annealed 5′-end immobilized first and second single stranded nucleic acid molecules as templates to generate double stranded nucleic acid molecules immobilized at both 5′-ends; and
vii) repeating steps iv) through vi) to generate multiple copies of the nucleic acid molecules on said planar solid surface, wherein steps iv) through vi) are carried out at the same temperature.

According to a second aspect of the invention, the method provides a means for generating multiple colonies or clusters of polynucleotide sequences which are copies of different single stranded polynucleotide molecules which possess common sequences at their 5′ and 3′ ends.

As described in detail herein, the present invention is directed to a method for amplifying a single stranded polynucleotide molecule on a solid support, comprising the steps of:
(a) providing a solid support having immobilised thereon at least one single stranded polynucleotide molecule which comprises at least one primer binding region and a plurality of primer oligonucleotides complementary to the at least one primer binding region of the single stranded polynucleotide;
(b) contacting the at least one single stranded polynucleotide molecule and the plurality of primer oligonucleotides with a first suitable buffer to promote hybridisation of the at least one single stranded polynucleotide molecule to a primer oligonucleotide to form at least one complex;
(c) contacting the at least one complex of step (b) with a second suitable buffer and an enzyme with polymerase activity and performing an extension reaction to extend the primer oligonucleotide of the complex by sequential addition of nucleotides to generate an extension product complementary to the at least one single stranded polynucleotide molecule; and
(d) contacting the extension product and the at least one single stranded polynucleotide molecule with a third suitable buffer to separate the single stranded polynucleotide molecule from the extension product and produce single stranded molecules immobilised on the solid support;
   wherein the method is carried out at substantially isothermal temperature.

In an aspect of the invention, steps (b) to (d) are repeated at least once, which repetition effectuates an increase in the number of single stranded polynucleotide molecules immobilised to the solid support. In one aspect, steps (b) to (d) are repeated to form at least one cluster of single stranded polynucleotide molecules immobilised to the solid support.

As described herein, the first, second, and third suitable buffers may be exchanged between steps (b), (c), and (d). In one embodiment, the exchange of the first, second, and third suitable buffers comprises the step of applying a suitable buffer via at least one inlet and removing the suitable buffer via at least one outlet.

As described herein, a first suitable buffer is a buffer that promotes or facilitates a hybridization reaction. Such hybridisation buffers, for example SSC or Tris HCl (at appropriate concentrations) are described herein and known in the art. A second suitable buffer is a buffer compatible with a polymerase extension reaction, which may comprise the hybridisation buffer plus additional components such as DNA polymerase and nucleoside triphoshates. Such polymerase extension buffers are described herein and known in the art. A third suitable buffer of the invention promotes nucleic acid denaturation. Denaturing buffers, for example sodium hydroxide or formamide (at appropriate concentrations) are described herein and known in the art.

### BRIEF DESCRIPTION OF THE DRAWINGS

FIG. 1A illustrates amplification of a single stranded polynucleotide molecule immobilised to a solid support.

FIG. 1B illustrates immobilisation of a single stranded polynucleotide molecule by hybridisation to and extension of a complementary primer immobilised to a solid support.

FIG. 2 illustrates amplification cycling using immobilised primers and single stranded polynucleotides in a method to produce clusters.

FIGS. 3A-3H demonstrate the use of 6 different enzymes in the method according to the invention. Isothermal amplification was carried out at 37° C. using Taq Polymerase, Bst Polymerase, Klenow, Pol I, T7 and T4 Polymerase for 30 cycles of amplification. Clusters stained using SYBR Green-I are clearly visible following amplification using Bst Polymerase (b) and Klenow (e).

FIGS. 4A-4F show a comparison of Bst Polymerase and Klenow in isothermal amplification according to the invention. At 37° C. Bst Polymerase produces more and brighter clusters.

FIGS. 5A and 5B depict results comparing the activity of Bst Polymerase (Channel 2) and Klenow (Channel 5) in the method according to the invention. Bst produced a greater number of clusters (N) (FIG. 5A) with an increased size (D) (FIG. 5B) relative to those produced by Klenow.

FIG. 5C compares Bst Polymerase (Channel 2) with Klenow (Channel 5) in the method according to the invention. Clusters amplified using Bst Polymerase exhibited a greater Filtered Cluster Intensity (I) when stained with SYBR Green-I than those amplified using Klenow.

FIG. 6 shows the monotemplate sequence of 240 bases SEQ ID NO: 1) used in the isothermal amplification process. Also shown in isolation are the sequences of 10T-P5 (SEQ ID NO: 2); SBS3 (SEQ ID NO: 3); and the reverse complement of 10T-P7 (SEQ ID NO: 4).

FIG. 7 shows a schematic representation of the hardware used to isothermally amplify a planar array. Surface amplification was carried out using an MJ Research thermocycler, coupled with an 8-way peristaltic pump Ismatec IPC ISM931 equipped with Ismatec tubing (orange/yellow, 0.51 mm ID).

### DETAILED DESCRIPTION OF THE INVENTION

The invention relates to a method of amplifying a single stranded polynucleotide molecule wherein said amplification is performed under conditions which are substantially isothermal.

The term “isothermal” refers to thermodynamic processes in which the temperature of a system remains constant: ΔT=0. This typically occurs when a system is in contact with an outside thermal reservoir (for example, heat baths and the like), and processes occur slowly enough to allow the system to continually adjust to the temperature of the reservoir through heat exchange.

The term “substantially isothermal” as used herein is therefore intended to mean that the system is maintained at essentially the same temperature. The term is also intended to capture minor deviations in temperature which might occur as the system equilibrates, for example when components which are of lower or higher temperature are added to the system. Thus it is intended that the term includes minor deviations from the temperature initially chosen to perform the method and those in the range of deviation of commercial thermostats. More particularly, the temperature deviation will be no more than about +/−2° C., more particularly no more than about +/−1° C., yet more particularly no more than about +/−0.5° C., no more than about +/−0.25° C., no more than about +/−0.1° C. or no more than about +/−0.01° C.

The term “amplifying” as used herein is intended to mean the process of increasing the numbers of a template polynucleotide sequence by producing copies. Accordingly it will be clear that the amplification process can be either exponential or linear. In exponential amplification the number of copies made of the template polynucleotide sequence increases at an exponential rate. For example, in an ideal PCR reaction with 30 cycles, 2 copies of template DNA will yield 2³⁰ or 1,073,741,824 copies. In linear amplification the number of copies made of the template polynucleotide sequences increases at a linear rate. For example, in an ideal 4-hour linear amplification reaction whose copying rate is 2000 copies per minute, one molecule of template DNA will yield 480,000 copies.

As used herein, the term “polynucleotide” refers to deoxyribonucleic acid (DNA), but where appropriate the skilled artisan will recognise that the method may also be applied to ribonucleic acid (RNA). The terms should be understood to include, as equivalents, analogs of either DNA or RNA made from nucleotide analogs. The term as used herein also encompasses cDNA, that is complementary or copy DNA produced from an RNA template, for example by the action of reverse transcriptase.

The single stranded polynucleotide molecules may have originated in single-stranded form, as DNA or RNA or may have originated in double-stranded DNA (dsDNA) form (e.g. genomic DNA fragments, PCR and amplification products and the like). Thus a single stranded polynucleotide may be the sense or antisense strand of a polynucleotide duplex. Methods of preparation of single stranded polynucleotide molecules suitable for use in the method of the invention using standard techniques are well known in the art. The precise sequence of the primary polynucleotide molecules is generally not material to the invention, and may be known or unknown.

In a particular embodiment, the single stranded polynucleotide molecules are DNA molecules. More particularly, the primary polynucleotide molecules represent the entire genetic complement of an organism, such as, for example a plant, bacteria, virus, or a mammal, and are genomic DNA molecules which include both intron and exon sequence (coding sequence), as well as non-coding regulatory sequences such as promoter and enhancer sequences. The present invention also encompasses use of particular sub-sets of polynucleotide sequences or genomic DNA, such as, for example, particular chromosomes. Yet more particularly, the sequence of the primary polynucleotide molecules is not known. Still yet more particularly, the primary polynucleotide molecules are human genomic DNA molecules.

The sequence of the primary polynucleotide molecules may be the same or different. A mixture of primary polynucleotide molecules of different sequences may, for example, be prepared by mixing a plurality (i.e., greater than one) of individual primary polynucleotide molecules. For example, DNA from more than one source can be prepared if each DNA sample is first tagged to enable its identification after it has been sequenced. Many different suitable DNA-tag methodologies exist in the art, as described in WO05068656, for example, which is included herein by reference, and are well within the purview of the skilled person.

The single stranded polynucleotide molecules to be amplified (referred to herein as templates) can originate as duplexes or single strands. For ease of reference, single stranded templates are described herein, since the duplexes need to be denatured prior to amplification. When viewed as a single strand, the 5′ ends and the 3′ ends of one strand of the template duplex may comprise different sequences, herein depicted as Y and Z for ease of reference. The other strand will be amplified in any isothermal amplification reaction, but would comprise sequence X at the 5‘end and Y’ at the 3′ end, where X is the complement of Z, and Y′ is the complement of Y. This strand may be present in many or all of the processes described herein, but is not further discussed.

In a particular embodiment, the single stranded polynucleotide molecule has two regions of known sequence. Yet more particularly, the regions of known sequence will be at the 5′ and 3′ termini of the single stranded polynucleotide molecule such that the single stranded polynucleotide molecule will be of the structure:

5[known sequence I]-[target polynucleotide sequence]-[known sequence II]-3′.

Typically “known sequence I” and “known sequence II” will consist of more than 20, or more than 40, or more than 50, or more than 100, or more than 300 consecutive nucleotides. The precise length of the two sequences may or may not be identical. Known sequence I may comprise a region of sequence Y, which may also be the sequence of one of the immobilised primers. Known sequence II may comprise a region of sequence Z, which hybridises to sequence X, which may be the sequence of another of the immobilised primers (a first primer, for example). Known sequences I and II may be longer than sequences Y and Z used to hybridise to the immobilised amplification primers.

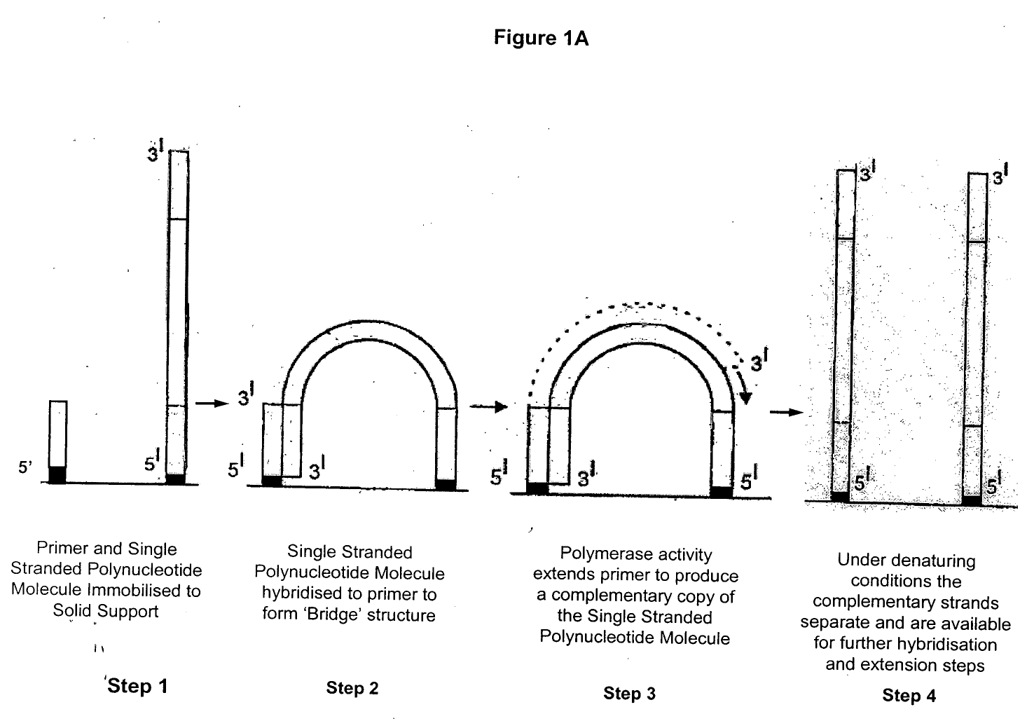

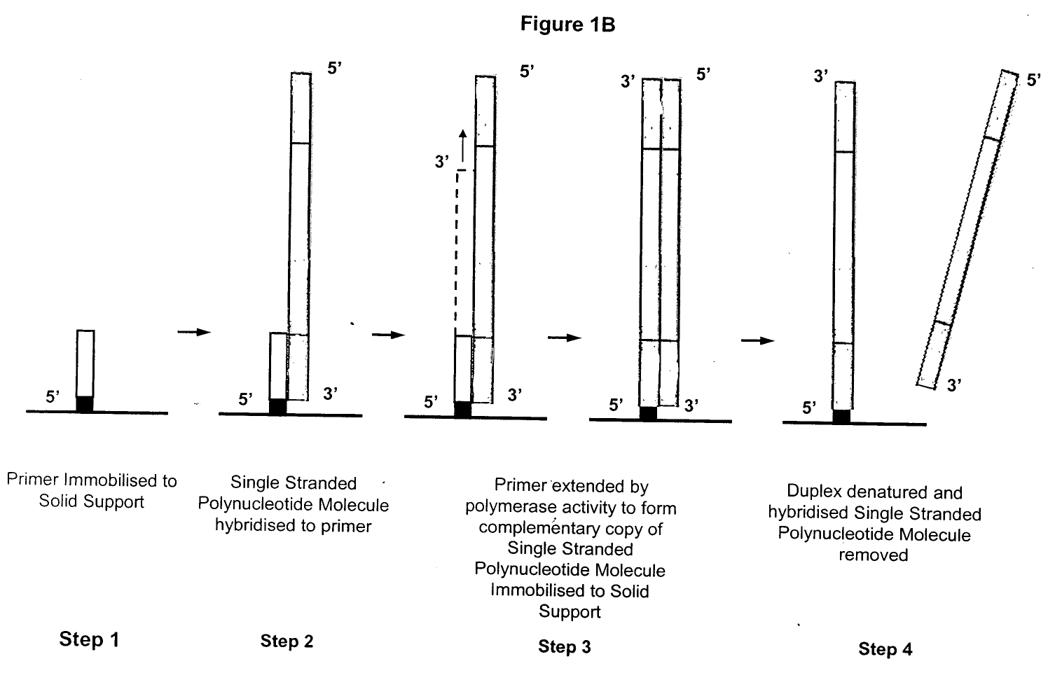

In a first step, a solid support having immobilised thereon said single stranded polynucleotide molecules and a plurality of primer oligonucleotides is provided. FIGS. 1A and 1B illustrate two embodiments whereby a single stranded polynucleotide molecule is immobilised directly to a solid support [1A] or is immobilised via hybridisation to and extension of a complementary primer immobilised to a solid support [1B].

The term “immobilised” as used herein is intended to encompass direct or indirect, covalent or non-covalent attachment, unless indicated otherwise, either explicitly or by context. In certain embodiments of the invention covalent attachment may be preferred, but generally all that is required is that the molecules (e.g. nucleic acids) remain immobilised or attached to a support under conditions in which it is intended to use the support, for example in applications requiring nucleic acid amplification and/or sequencing.

The term “solid support” as used herein refers to any inert substrate or matrix to which nucleic acids can be attached, such as for example latex beads, dextran beads, polystyrene surfaces, polypropylene surfaces, polyacrylamide gel, gold surfaces, glass surfaces and silicon wafers. The solid support may be a glass surface. The solid support may further be a planar surface, although the invention may also be performed on beads which are moved between containers of different buffers, or beads arrayed on a planar surface.

In certain embodiments the solid support may comprise an inert substrate or matrix which has been “functionalised”, for example by the application of a layer or coating of an intermediate material comprising reactive groups which permit covalent attachment to molecules such as polynucleotides. By way of non-limiting example such supports may include polyacrylamide hydrogels supported on an inert substrate such as glass. In such embodiments the molecules (polynucleotides) may be directly covalently attached to the intermediate material (e.g. the hydrogel), but the intermediate material may itself be non-covalently attached to the substrate or matrix (e.g. the glass substrate). Such an arrangement is described more fully in co-pending application WO 05065814, whose contents are included herein by reference, and covalent attachment to a solid support is to be interpreted accordingly as encompassing this type of arrangement.

Primer oligonucleotides or primers are polynucleotide sequences that are capable of annealing specifically to the single stranded polynucleotide template to be amplified under conditions encountered in the primer annealing step of each cycle of an amplification reaction. Generally amplification reactions require at least two amplification primers, often denoted “forward” and “reverse” primers. In certain embodiments the forward and reverse primers may be identical. The forward primer oligonucleotides must include a “template-specific portion”, being a sequence of nucleotides capable of annealing to a primer-binding sequence in one strand of the molecule to be amplified and the reverse primer oligonucleotides must include a template specific portion capable of annealing to the complement of that strand during the annealing step. The primer binding sequences generally will be of known sequence and will therefore particularly be complementary to a sequence within known sequence I and/or known sequence II of the single stranded polynucleotide molecule. The length of the primer binding sequences Y and Z need not be the same as those of known sequence I or II, and are preferably shorter, being particularly 16-50 nucleotides, more particularly 16-40 nucleotides and yet more particularly 20-30 nucleotides in length. The optimum length of the primer oligonucleotides will depend upon a number of factors and it is preferred that the primers are long (complex) enough so that the likelihood of annealing to sequences other than the primer binding sequence is very low.

Generally primer oligonucleotides are single stranded polynucleotide structures. They may also contain a mixture of natural and non-natural bases and also natural and non-natural backbone linkages, provided that any non-natural modifications do not preclude function as a primer—that being defined as the ability to anneal to a template polynucleotide strand during conditions of the amplification reaction and to act as an initiation point for synthesis of a new polynucleotide strand complementary to the template strand.

Primers may additionally comprise non-nucleotide chemical modifications, again provided such that modifications do not prevent primer function. Chemical modifications may, for example, facilitate covalent attachment of the primer to a solid support. Certain chemical modifications may themselves improve the function of the molecule as a primer, or may provide some other useful functionality, such as providing a site for cleavage to enable the primer (or an extended polynucleotide strand derived therefrom) to be cleaved from a solid support.

Although the invention may encompass “solid-phase amplification” methods in which only one amplification primer is immobilised (the other primer usually being present in free solution), in a particular embodiment, the solid support may be provided with both the forward and reverse primers immobilised. In practice there will be a plurality of identical forward primers and/or a plurality of identical reverse primers immobilised on the solid support, since the amplification process requires an excess of primers to sustain amplification. Thus references herein to forward and reverse primers are to be interpreted accordingly as encompassing a plurality of such primers unless the context indicates otherwise.

“Solid-phase amplification” as used herein refers to any nucleic acid amplification reaction carried out on or in association with a solid support such that all or a portion of the amplified products remain immobilised on the solid support as they are formed. In particular the term encompasses solid phase amplification reactions analogous to standard solution phase PCR except that one or both of the forward and reverse amplification primers is/are immobilised on the solid support.

As will be appreciated by the skilled reader, any given amplification reaction usually requires at least one type of forward primer and at least one type of reverse primer specific for the template to be amplified. However, in certain embodiments the forward and reverse primers may comprise template specific portions of identical sequence, and may have entirely identical nucleotide sequence and structure (including any non-nucleotide modifications). In other words, it is possible to carry out solid phase amplification using only one type of primer, and such single primer methods are encompassed within the scope of the invention. Other embodiments may use forward and reverse primers which contain identical template-specific sequences but which differ in some other structural features. For example, one type of primer may contain a non-nucleotide modification which is not present in the other. In still yet another embodiment the template-specific sequences are different and only one primer is used in a method of linear amplification.

In other embodiments of the invention the forward and reverse primers may contain template-specific portions of different sequence.

In all embodiments of the invention, amplification primers for solid phase amplification are immobilised by single point covalent attachment to the solid support at or near the 5′ end of the primer, leaving the template-specific portion of the primer free to anneal to its cognate template and the 3′ hydroxyl group free to function in primer extension. The chosen attachment chemistry will depend on the nature of the solid support, and any functionalisation or derivatisation applied to it. The primer itself may include a moiety, which may be a non-nucleotide chemical modification to facilitate attachment. In one particular embodiment the primer may include a sulphur containing nucleophile such as phosphoriothioate or thiophosphate at the 5′ end. In the case of solid supported polyacrylamide hydrogels, this nucleophile will bind to a bromoacetamide group present in the hydrogel.

In a particular embodiment the means of attaching the primers to the solid support is via 5′ phosphorothioate attachment to a hydrogel comprised of polymerised acrylamide and N-(5-bromoacetamidylpentyl) acrylamide (BRAPA). Such an arrangement is described more fully in co-pending application WO 05065814, which is incorporated herein by reference in its entirety.

The single stranded polynucleotide molecule is immobilised to the solid support at or near the 5′ end. The chosen attachment chemistry will depend on the nature of the solid support, and any functionalisation or derivitisation applied to it. The single stranded polynucleotide molecule itself may include a moiety, which may be a non-nucleotide chemical modification to facilitate attachment. In one particular embodiment, the single stranded polynucleotide molecule may include a sulphur containing nucleophile such as phosphoriothioate or thiophosphate at the 5′ end. In the case of solid supported polyacrylamide hydrogels, this nucleophile will also bind to the bromoacetamide groups present in the hydrogel.

In one embodiment the means of attaching the single stranded polynucleotide molecule to the solid support is via 5′ phosphorothioate attachment to a hydrogel comprised of polymerised acrylamide and N-(5-bromoacetamidylpentyl)acrylamide (BRAPA).

The single stranded polynucleotide molecule and primer oligonucleotides of the invention are mixed together in appropriate proportions so that when they are attached to the solid support an appropriate density of attached single stranded polynucleotide molecules and primer oligonucleotides is obtained. Preferably the proportion of primer oligonucleotides in the mixture is higher than the proportion of single stranded polynucleotide molecules. Preferably the ratio of primer oligonucleotides to single stranded polynucleotide molecules is such that when immobilised to the solid support, a “lawn” of primer oligonucleotides is formed comprising a plurality of primer oligonucleotides being located at an approximately uniform density over the whole or a defined area of the solid support, with one or more single stranded polynucleotide molecule(s) being immobilised individually at intervals within the lawn of primer oligonucleotides.

The distance between the individual primer oligonucleotides and the one or more single stranded polynucleotide molecules (and hence the density of the primer oligonucleotides and single stranded polynucleotide molecules) can be controlled by altering the concentration of primer oligonucleotides and single stranded polynucleotide molecules that are immobilised to the support. A preferred density of primer oligonucleotides is at least 1 fmol/mm², preferably at least 10 fmol/mm², more preferably between 30 to 60 fmol/mm². The density of single stranded polynucleotide molecules for use in the method of the invention is typically 10,000/mm² to 100,000/mm². Higher densities, for example, 100,000/mm² to 1,000,000/mm² and 1,000,000/mm² to 10,000,000/mm² may also be achieved.

Controlling the density of attached single stranded polynucleotide molecules and primer oligonucleotides in turn allows the final density of nucleic acid colonies on the surface of the support to be controlled. This is due to the fact that according to the method of the invention, one nucleic acid colony can result from the attachment of one single stranded polynucleotide molecule, providing the primer oligonucleotides of the invention are present in a suitable location on the solid support. The density of single stranded polynucleotide molecules within a single colony can also be controlled by controlling the density of attached primer oligonucleotides.

In another embodiment, a complementary copy of the single stranded polynucleotide molecule is attached to the solid support by a method of hybridisation and primer extension. Methods of hybridisation for formation of stable duplexes between complementary sequences by way of Watson-Crick base-pairing are known in the art. The single stranded template may originate from a duplex that has been denatured in solution, for example by sodium hydroxide or formamide treatment and then diluted into hybridisation buffer. The template may be hybridised to the surface at a temperature different to that used for subsequent amplification cycles. The immobilised primer oligonucleotides hybridise at and are complementary to a region or template specific portion of the single stranded polynucleotide molecule. An extension reaction may then be carried out wherein the primer is extended by sequential addition of nucleotides to generate a complementary copy of the single stranded polynucleotide sequence attached to the solid support via the primer oligonucleotide. The single stranded polynucleotide sequence not immobilised to the support may be separated from the complementary sequence under denaturing conditions and removed, for example by washing with hydroxide or formamide. The primer used for the initial primer extension of a hybridised template may be one of the forward or reverse primers used in the amplification process. After an initial hybridisation, extension and separation, an immobilised template strand is obtained.

The terms “separate” and “separating” are broad terms which refer primarily to the physical separation of the DNA bases that interact within, for example, a Watson-Crick DNA-duplex of the single stranded polynucleotide sequence and its complement. The terms also refer to the physical separation of both of these strands. In their broadest sense the terms refer to the process of creating a situation wherein annealing of another primer oligonucleotide or polynucleotide sequence to one of the strands of a duplex becomes possible.

Accordingly it will be appreciated that in the case where a single stranded polynucleotide molecule has reacted with the surface and is attached, the result will be the same as in the case when the strand is hybridised and one amplification step has been performed to provide a complementary single stranded polynucleotide molecule attached to the surface.

In yet another embodiment the single stranded polynucleotide molecule is ligated to primers immobilised to the solid support using ligation methods known in the art and standard methods (Sambrook and Russell, Molecular Cloning, A Laboratory Manual, third edition). Such methods utilise ligase enzymes such as DNA ligase to effect or catalyse joining of the ends of the two polynucleotide strands of, in this case, the single stranded polynucleotide molecule and the primer oligonucleotide such that covalent linkages are formed. In this context, joining means covalent linkage of two polynucleotide strands which were not previously covalently linked.

In a particular aspect of the invention, such joining takes place by formation of a phosphodiester linkage between the two polynucleotide strands, but other means of covalent linkage (e.g. non-phosphodiester backbone linkages) may be used. Another equally applicable method is splicing by overlap extension (SOE). In SOE polynucleotide molecules are joined at precise junctions irrespective of nucleotide sequences at the recombination site and without the use of restriction endonucleases or ligase. Fragments from the polynucleotide molecules that are to be recombined are generated by methods known in the art. The primers are designed so that the ends of the products contain complementary sequences. When these polynucleotide molecules are mixed, denatured, and reannealed, the strands having the matching sequences at their 3′ ends overlap and act as primers for each other. Extension of this overlap by DNA polymerase produces a molecule in which the original sequences are ‘spliced’ together. The method originally disclosed by Horton et al (Gene. 1989 Apr. 15; 77(1):61-8) may also potentially be performed isothermally.

Once the primer oligonucleotides and single stranded polynucleotide molecules of the invention have been immobilised on the solid support at the appropriate density, extension products can then be generated by carrying out an appropriate number of cycles of amplification on the covalently bound single stranded polynucleotide molecules so that each colony, or cluster comprises multiple copies of the original immobilised single stranded polynucleotide molecule (and its complementary sequence). One cycle of amplification consists of the steps of hybridisation, extension and denaturation and these steps are generally comparable with the steps of hybridisation, extension and denaturation of PCR with the exception that in the present invention each step is performed at substantially isothermal temperature. Suitable reagents for performing the method according to the invention are well known in the art.

Thus in a next step according to the present invention suitable conditions are applied to the single stranded polynucleotide molecule and the plurality of primer oligonucleotides such that sequence Z at the 3′ end of the single stranded polynucleotide molecule hybridises to a primer oligonucleotide sequence X to form a complex wherein, the primer oligonucleotide hybridises to the single stranded template to create a ‘bridge’ structure.

Suitable conditions such as neutralising and/or hybridising buffers are well known in the art (See Sambrook et al., Molecular Cloning, A Laboratory Manual, 3^{rd} Ed, Cold Spring Harbor Laboratory Press, NY; Current Protocols, eds Ausubel et al.). The neutralising and/or hybridising buffer may then be removed. A suitable hybridisation buffer is referred to as ‘amplification pre-mix’, and contains 2 M betaine, 20 mM Tris, 10 mM Ammonium Sulfate, 2 mM Magnesium sulfate, 0.1% Triton, 1.3% DMSO, pH 8.8.

Next, by applying suitable conditions for extension, an extension reaction is performed. The primer oligonucleotide of the complex is extended by sequential addition of nucleotides to generate an extension product complementary to the single stranded polynucleotide molecule.

Suitable conditions such as extension buffers/solutions comprising an enzyme with polymerase activity are well known in the art (See Sambrook et al., Molecular Cloning, A Laboratory Manual, 3^{rd} Ed, Cold Spring Harbor Laboratory Press, NY; Current Protocols, eds Ausubel et al.). In a particular embodiment dNTP's may be included in the extension buffer. In a further embodiment dNTP's could be added prior to the extension buffer.

Examples of enzymes with polymerase activity which can be used in the present invention are DNA polymerase (Klenow fragment, T4 DNA polymerase), heat-stable DNA polymerases from a variety of thermostable bacteria (such as Taq, VENT, Pfu, Tfl DNA polymerases) as well as their genetically modified derivatives (TaqGold, VENTexo, Pfu exo). A combination of RNA polymerase and reverse transcriptase can also be used to generate the extension products. Particularly the enzyme has strand displacement activity, more particularly the enzyme will be active at a pH of about 7 to about 9, particularly pH 7.9 to pH 8.8, yet more particularly the enzymes are Bst or Klenow.

In one embodiment, the nucleoside triphosphate molecules used are deoxyribonucleotide triphosphates, for example DATP, dTTP, dCTP, dGTP, or are ribonucleoside triphosphates for example ATP, UTP, CTP, GTP. The nucleoside triphosphate molecules may be naturally or non-naturally occurring. The amplification buffer may also contain additives such as DMSO and or betaine to normalise the melting temperatures of the different sequences in the template strands. A suitable solution for extension is referred to as ‘amplification mix’ and contains 2 M betaine, 20 mM Tris, 10 mM Ammonium Sulfate, 2 mM Magnesium sulfate, 0.1% Triton, 1.3% DMSO, pH 8.8 plus 200 μM dNTP's and 80 units/mL of Bst polymerase (NEB Product ref M0275L).

After the hybridisation and extension steps, the support and attached nucleic acids are subjected to denaturation conditions. Preferably the extension buffer is first removed. Suitable denaturing buffers are well known in the art (See Sambrook et al., Molecular Cloning, A Laboratory Manual, 3^{rd} Ed, Cold Spring Harbor Laboratory Press, NY; Current Protocols, eds. Ausubel et al.). By way of example it is known that alterations in pH and low ionic strength solutions can denature nucleic acids at substantially isothermal temperatures. Formamide and urea form new hydrogen bonds with the bases of nucleic acids, thereby disrupting hydrogen bonds that lead to Watson-Crick base pairing. In a particular embodiment the concentration of formamide is 50% or more, and may be used neat. Such conditions result in denaturation of double stranded nucleic acid molecules to single stranded nucleic acid molecules. Alternatively the strands may be separated by treatment with a solution of very low salt (for example less than 0.1 mM cationic conditions) and high pH (>12) or by using a chaotropic salt (e.g. guanidinium hydrochloride). In a particular embodiment a strong base may be used. A strong base is a basic chemical compound that is able to deprotonate very weak acids in an acid base reaction. The strength of a base is indicated by its pK_{b} value, compounds with a pK_{b} value of less than about 1 are called strong bases and are well known to a skilled practitioner. In a particular embodiment the strong base is Sodium Hydroxide (NaOH) solution used at a concentration of from 0.05M to 0.25M. More particularly NaOH is used at a concentration of 0.1M.

Following denaturation, two immobilised nucleic acids are produced from a double stranded nucleic acid molecule, the first being the initial immobilised single stranded polynucleotide template molecule and the second being a nucleic acid complementary thereto, extending from one of the immobilised primer oligonucleotides, comprising sequence X at the 5′ end. Both the original immobilised single stranded polynucleotide molecule and the immobilised extended primer oligonucleotide formed are then able to initiate further rounds of amplification on subjecting the support to further cycles of hybridisation, extension and denaturation by hybridisation to primer sequences X and Y respectively.

It may be advantageous to perform optional washing steps in between each step of the amplification method. For example an extension buffer without polymerase enzyme with or without dNTP's could be applied to the solid support before being removed and replaced with complete extension buffer (extension buffer that includes all necessary components for extension to proceed).

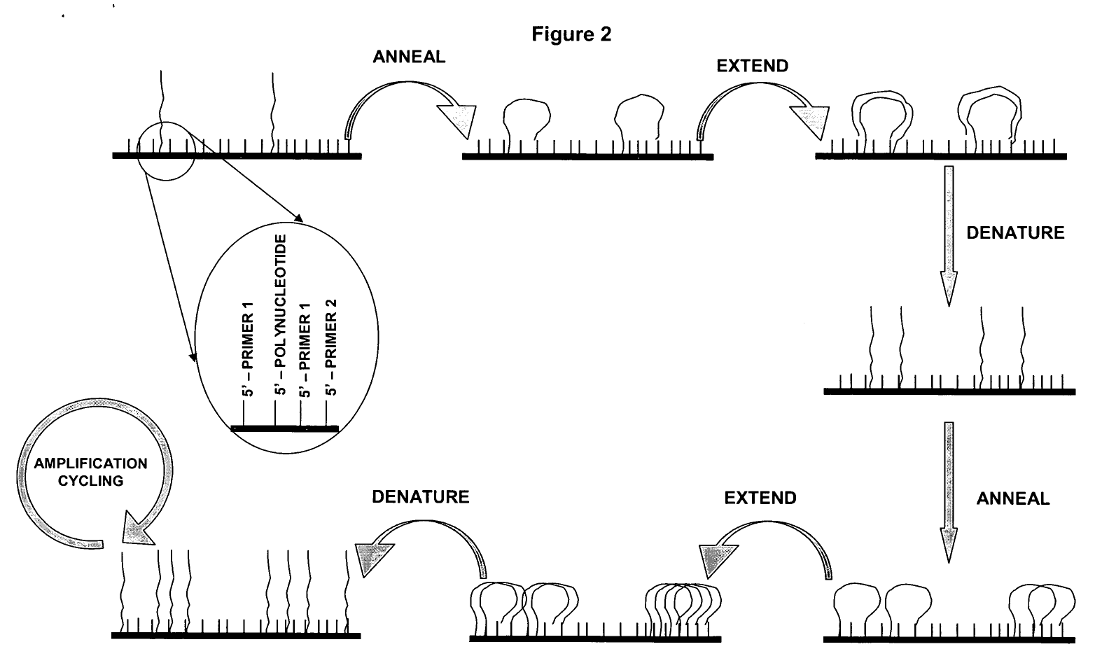

Such further rounds of amplification result in a nucleic acid colony or “cluster” comprising multiple immobilised copies of the single stranded polynucleotide sequence and its complementary sequence. See FIG. 2, which illustrates amplification cycling using immobilised primers and single stranded polynucleotides in a method to produce clusters.

The initial immobilisation of the single stranded polynucleotide molecule means that the single stranded polynucleotide molecule can only hybridise with primer oligonucleotides located at a distance within the total length of the single stranded polynucleotide molecule.

Thus, the boundary of the nucleic acid colony or cluster formed is limited to a relatively local area, namely the area in which the initial single stranded polynucleotide molecule was immobilised. As the templates and the complementary copies thereof remain immobilised throughout the whole amplification process, the templates do not intermingle, unless the clusters are amplified to an extent whereby they become large enough to overlap on the surface. The absence of non-immobilised nucleic acids throughout the amplification process, therefore, prevents diffusion of the templates, which can initiate additional clusters elsewhere on the surface.

Clearly, once more copies of the single stranded polynucleotide molecule and its complement have been synthesised by carrying out further rounds of amplification, i.e., further rounds of hybridisation, extension and denaturation, then the boundary of the nucleic acid colony or cluster being generated is extended further, although the boundary of the colony formed is still limited to a relatively localised area, essentially in the vicinity of the area in which the initial single stranded polynucleotide molecule was immobilised. Clusters may be of a diameter of 100 nm to 10 μm, a higher information density being obtainable from a clustered array where the clusters are of a smaller size.

It can thus be seen that the method of the present invention allows for the generation of a nucleic acid colony from a single immobilised single stranded polynucleotide molecule and that the size of these colonies can be controlled by altering the number of rounds of amplification to which the single stranded polynucleotide molecule is subjected.

An essential feature of the invention is that the hybridisation, extension and denaturation steps are all carried out at the same, substantially isothermal temperature. In a particular embodiment, the temperature is from 37° C. to about 75° C., depending on the choice of enzyme, more particularly from 50° C. to 70° C., and yet more particularly from 60° C. to 65° C. for Bst polymerase. In a particular embodiment the substantially isothermal temperature may be the around the melting temperature of the oligonucleotide primer(s). Methods of calculating appropriate melting temperatures are known in the art. For example the annealing temperature may be about 5° C. below the melting temperature (Tm) of the oligonucleotide primers. In yet another particular embodiment the substantially isothermal temperature may be determined empirically and is the temperature at which the oligonucleotide displays greatest specificity for the primer binding site whilst reducing non-specific binding.

In contrast to prior art isothermal methods, the instant method has the surprising advantage that even at lower temperatures, such as, for example 37° C., specificity of primer binding is maintained. Not wishing to be bound by hypothesis, it is believed that where primers and polynucleotide sequences are both immobilised to a solid support, the potential for mis-priming is reduced. For example, in solution-based amplification the primers are potentially able to bind incorrectly at regions over the entire length of the template sequence. In controlling the density of immobilised primer and template sequence, the availability of sequences which the primers can effectively ‘reach’ is reduced, possibly favouring binding to the primer binding sites at the termini of the single stranded polynucleotide sequences even in conditions of low stringency, i.e. lower temperatures.

The present inventors have also discovered that carrying out substantially isothermal amplification by changing solutions in contact with the solid support has the additional advantage of producing clusters containing higher levels of nucleic acid than are achieved using for example, conventional thermally cycled amplification. Again, not wishing to be bound by hypothesis, it is believed that under thermal cycling conditions more attachments between the immobilised nucleic acids and the solid support are broken. This results in a loss of primer oligonucleotides, single stranded polynucleotide molecules and extension products from the solid support. During conventional thermal cycling in a ‘sealed’ system there is also a net loss of polymerase enzyme activity, which further reduces efficiency of the amplification.

These problems are overcome by performing solid-phase amplification under substantially isothermal conditions, and not heating to high temperatures such as 95° C. for example. Changing the solutions in contact with the solid support renews not only the components of the reactions which may be rate limiting, such as the enzyme or dNTPs, but also results in greater stability of the surface (and surface chemistry) and ‘brighter’ clusters during downstream sequencing.

Thus the number of nucleic acid colonies or clusters formed on the surface of the solid support is dependent upon the number of single stranded polynucleotide molecules which are initially immobilised to the support, providing there are a sufficient number of immobilised primer oligonucleotides within the locality of each immobilised single stranded polynucleotide molecule. It is for this reason that the solid support to which the primer oligonucleotides and single stranded polynucleotide molecules have been immobilised may comprise a lawn of immobilised primer oligonucleotides at an appropriate density with single stranded polynucleotide molecules immobilised at intervals within the lawn of primers. The density of the templates may be the same density of clusters, namely 10⁴-10⁷/mm², said density being capable of individual optical resolution of the individual molecules.

In a particular aspect, the method according to the first aspect of the invention is used to prepare clustered arrays of nucleic acid colonies, analogous to those described in WO 00/18957 or WO 98/44151 (the contents of which are herein incorporated by reference), by solid-phase amplification under substantially isothermal conditions. The terms “cluster” and “colony” are used interchangeably herein to refer to a discrete site on a solid support comprised of a plurality of identical immobilised nucleic acid strands and a plurality of identical immobilised complementary nucleic acid strands. The term “clustered array” refers to an array comprising such clusters or colonies. In this context the term “array” is not to be understood as requiring an ordered arrangement of clusters.

Use in Substantially Isothermal Amplification of Libraries

In a further aspect, the invention provides a method of solid-phase nucleic acid amplification of a 5′ and 3′ modified library of template polynucleotide molecules which have common sequences at their 5′ and 3′ ends, wherein a solid-phase nucleic acid amplification reaction is performed under substantially isothermal conditions to amplify said template polynucleotide molecules.

In this context the term “common” is interpreted as meaning common to all templates in the library. As explained in further detail herein, all templates within the 5′ and 3′ modified library will contain regions of common sequence Y and Z at (or proximal to) their 5′ and 3′ ends, particularly wherein the common sequence at the 5′ end of each individual template in the library is not identical and not fully complementary to the common sequence at the 3′ end of said template. The term “5′ and 3′ modified library” refers to a collection or plurality of template molecules which share common sequences at their 5′ ends and common sequences at their 3′ ends. Use of the term “5′ and 3′ modified library” to refer to a collection or plurality of template molecules should not be taken to imply that the templates making up the library are derived from a particular source, or that the “5′ and 3′ modified library” has a particular composition. By way of example, use of the term “5′ and 3′ modified library” should not be taken to imply that the individual templates within the library must be of different nucleotide sequence or that the templates be related in terms of sequence and/or source.

In its various embodiments the invention encompasses use of so-called “mono-template” libraries, which comprise multiple copies of a single type of template molecule, each having common sequences at their 5′ ends and their 3′ ends, as well as “complex” libraries wherein many, if not all, of the individual template molecules comprise different target sequences (as defined below), although all share common sequences at their 5′ ends and 3′ ends. Such complex template libraries may be prepared from a complex mixture of target polynucleotides such as (but not limited to) random genomic DNA fragments, cDNA libraries, etc. The invention may also be used to amplify “complex” libraries formed by mixing together several individual “mono-template” libraries, each of which has been prepared separately starting from a single type of target molecule (i.e., a mono-template). In particular embodiments, more than 50%, or more than 60%, or more than 70%, or more than 80%, or more than 90%, or more than 95% of the individual polynucleotide templates in a complex library may comprise different target sequences, although all templates in a given library will share common sequence at their 5′ ends and common sequence at their 3′ ends.

Use of the term “template” to refer to individual polynucleotide molecules in the library indicates that one or both strands of the polynucleotides in the library are capable of acting as templates for template dependent nucleic acid polymerisation catalysed by a polymerase. Use of this term should not be taken as limiting the scope of the invention to libraries of polynucleotides which are actually used as templates in a subsequent enzyme-catalysed polymerisation reaction. Each strand of each template molecule in the library should have the following structure, when viewed as a single strand:

5′-[known sequence I]-[target sequence]-[known sequence II]-3′.

Wherein “known sequence I” is common to all template molecules in the library; “target sequence” represents a sequence which may be different in different individual template molecules within the library; and “known sequence II” represents a sequence also common to all template molecules in the library. Known sequences I and II will also include “primer binding sequence Y” and “primer binding sequence Z” and since they are common to all template strands in the library they may include “universal” primer-binding sequences, enabling all templates in the library to be ultimately amplified in a solid-phase amplification procedure using universal primers comprising sequences X and Y, where X is complementary to Z. It is a key feature of the invention, however, that the common 5′ and 3′ end sequences denoted “known sequence I” and “known sequence II” are not fully complementary to each other, meaning that each individual template strand can contain different (and non-complementary) universal primer sequences at its 5′ and 3′ ends. It is generally advantageous for complex libraries of templates to be amplified by solid phase amplification to include regions of “different” sequence at their 5′ and 3′ ends, which are nevertheless common to all template molecules in the library, especially if the amplification products are to be sequenced ultimately. For example, the presence of a common unique sequence at one end only of each template in the library can provide a binding site for a sequencing primer, enabling one strand of each template in the amplified form of the library to be sequenced in a single sequencing reaction using a single type of sequencing primer.

In a particular embodiment, the library is a library of single stranded polynucleotide molecules. Where the library comprises polynucleotide molecule duplexes, methods for preparing single stranded polynucleotide molecules from the library are known in the art. For example the library may be heated to a suitable temperature, or treated with hydroxide or formamide, to separate each strand of the duplexes before carrying out the method according to the invention. In another embodiment one strand of the duplex may have a modification, such as, for example biotin. Following strand separation by appropriate methods, the biotinylated strands can be separated from the complementary strands, using for example avidin coated micro-titre plates and the like, to effectively produce two single stranded populations or libraries. Thus the method according to the invention is as applicable to one single stranded polynucleotide molecule as it is to a plurality of single stranded polynucleotide molecules.

In yet another embodiment, more than two, for example, three, four, or more than four different primer oligonucleotides may be grafted to the solid support. In this manner more than one library, with common sequences that differ between the libraries (wherein common sequences attached thereto are specific for each library), may be isothermally amplified, such as, for example libraries prepared from two different patients.

Use in Sequencing/Methods of Sequencing

The invention also encompasses methods of sequencing amplified nucleic acids generated by isothermal solid-phase amplification. Thus, the invention provides a method of nucleic acid sequencing comprising amplifying a 5′ and 3′ modified library of nucleic acid templates using isothermal solid-phase amplification as described above and carrying out a nucleic acid sequencing reaction to determine the sequence of the whole or a part of at least one amplified nucleic acid strand produced in the solid-phase amplification reaction.

Sequencing can be carried out using any suitable sequencing technique, wherein nucleotides are added successively to a free 3′ hydroxyl group, resulting in synthesis of a polynucleotide chain in the 5′ to 3′ direction. The nature of the nucleotide added may be determined after each nucleotide addition. Sequencing techniques using sequencing by ligation, wherein not every contiguous base is sequenced, and techniques such as massively parallel signature sequencing (MPSS) where bases are removed from, rather than added to the strands on the surface are also within the scope of the invention, as are techniques using detection of pyrophosphate release (pyrosequencing). Such pyrosequencing based techniques are particularly applicable to sequencing arrays of beads where the beads have been isothermally amplified and where a single template from the library molecule is amplified on each bead.

The initiation point for the sequencing reaction may be provided by annealing of a sequencing primer to a product of the isothermal solid-phase amplification reaction. In this connection, one or both of the adapters added during formation of the template 5′ and 3′ modified library may include a nucleotide sequence which permits annealing of a sequencing primer to amplified products derived from the isothermal solid-phase amplification of the template 5′ and 3′ modified library.

The products of solid-phase amplification reactions wherein both forward and reverse amplification primers are covalently immobilised on the solid surface are so-called “bridged” structures formed by annealing of pairs of immobilised polynucleotide strands and immobilised complementary strands, both strands being attached to the solid support at the 5′ end. Arrays comprising such bridged structures may provide inefficient templates for nucleic acid sequencing, since hybridisation of a conventional sequencing primer to one of the immobilised strands is not favoured compared to annealing of this strand to its immobilised complementary strand under standard conditions for hybridisation.

In order to provide more suitable templates for nucleic acid sequencing, substantially all, or at least a portion of, one of the immobilised strands in the “bridged” structure may be removed in order to generate a template which is at least partially single-stranded. The portion of the template which is single-stranded will thus be available for hybridisation to a sequencing primer. The process of removing all or a portion of one immobilised strand in a “bridged” double-stranded nucleic acid structure may be referred to herein as “linearisation”.

Bridged template structures may be linearised by cleavage of one or both strands with a restriction endonuclease or by cleavage of one strand with a nicking endonuclease. Other methods of cleavage can be used as an alternative to restriction enzymes or nicking enzymes, including inter alia chemical cleavage (e.g. cleavage of a diol linkage with periodate), cleavage of abasic sites by cleavage with endonuclease, or by exposure to heat or alkali, cleavage of ribonucleotides incorporated into amplification products otherwise comprised of deoxyribonucleotides, photochemical cleavage or cleavage of a peptide linker. Methods of linearization are detailed in co-pending application WO07010251, the contents of which is included herein by reference in its entirety.

It will be appreciated that a linearization step may not be essential if the solid-phase amplification reaction is performed with only one primer covalently immobilised and the other in free solution.

In order to generate a linearised template suitable for sequencing it is necessary to remove the cleaved complementary strands in the bridged structure that remain hybridised to the uncleaved strand. This denaturing step is a part of the ‘linearisation process’, and can be carried out by standard techniques such as heat or chemical treatment with hydroxide or formamide solution. In a particular embodiment, one strand of the bridged structure is substantially or completely removed by the process of chemical cleavage and denaturation. Denaturation results in the production of a sequencing template which is partially or substantially single-stranded. A sequencing reaction may then be initiated by hybridisation of a sequencing primer to the single-stranded portion of the template.

Thus, the invention encompasses methods wherein the nucleic acid sequencing reaction comprises hybridising a sequencing primer to a single-stranded region of a linearised amplification product, sequentially incorporating one or more nucleotides into a polynucleotide strand complementary to the region of amplified template strand to be sequenced, identifying the base present in one or more of the incorporated nucleotide(s), or one or more of the bases present in the oligonucleotides, and thereby determining the sequence of a region of the template strand.

One particular sequencing method which can be used in accordance with the invention relies on the use of modified nucleotides having removable 3′ blocks, for example as described in WO04018497 and U.S. Pat. No. 7,057,026. Once the modified nucleotide has been incorporated into the growing polynucleotide chain complementary to the region of the template being sequenced there is no free 3′-OH group available to direct further sequence extension and therefore the polymerase can not add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination among the bases added during each incorporation step. Alternatively, a separate reaction may be carried out containing each of the modified nucleotides separately.

The modified nucleotides may carry a label to facilitate their detection. In a particular embodiment, this is a fluorescent label. Each nucleotide type may carry a different fluorescent label. However the detectable label need not be a fluorescent label. Any label can be used which allows the detection of an incorporated nucleotide.

One method for detecting fluorescently labelled nucleotides comprises using laser light of a wavelength specific for the labelled nucleotides, or the use of other suitable sources of illumination. The fluorescence from the label on the nucleotide may be detected by a CCD camera or other suitable detection means.

The invention is not intended to be limited to use of the sequencing method outlined above, as essentially any sequencing methodology which relies on successive incorporation or removal of nucleotides into or from a polynucleotide chain can be used. Suitable alternative techniques include, for example, Pyrosequencing™, FISSEQ (fluorescent in situ sequencing), MPSS (massively parallel signature sequencing) and sequencing by ligation-based methods, for example as described in U.S. Pat. No. 6,306,597.

The target polynucleotide to be sequenced using the method of the invention may be any polynucleotide that it is desired to sequence. Using the isothermal amplification method described in detail herein it is possible to prepare a clustered array of template libraries starting from essentially any double or single-stranded target polynucleotide of known, unknown or partially known sequence. With the use of clustered arrays prepared by solid-phase amplification it is possible to sequence multiple targets of the same or different sequence in parallel. Sequencing may result in determination of the sequence of a whole or a part of the target molecule.

Use of Clustered Arrays

Clustered arrays formed by the methods of the invention are suitable for use in applications usually carried out on ordered arrays such as micro-arrays. Such applications by way of non-limiting example include hybridisation analysis, gene expression analysis, protein binding analysis and the like. The clustered array may be sequenced before being used for downstream applications such as, for example, hybridisation with fluorescent RNA or binding studies using fluorescent labelled proteins.

Apparatus

Advantageously, substantially isothermal solid phase amplification can be performed efficiently in a flow cell since it is a key feature of the invention that the primers, template and amplified (extension) products all remain immobilised to the solid support and are not removed from the support at any stage during the substantially isothermal amplification.

Such an apparatus may include one or more of the following:

a) at least one inlet

b) means for immobilising primers on a surface (although this is not needed if immobilised primers are already provided);

c) means for substantially isothermal amplification of nucleic acids (e.g. denaturing solution, hybridising solution, extension solution, wash solution(s));

d) at least one outlet

e) control means for coordinating the different steps required for the method of the present invention.

Other apparatuses are within the scope of the present invention.

These allow immobilised nucleic acids to be isothermally amplified. They may also include a source of reactants and detecting means for detecting a signal that may be generated once one or more reactants have been applied to the immobilised nucleic acid molecules. They may also be provided with a surface comprising immobilised nucleic acid molecules in the form of colonies, as described supra.

In a preferred embodiment as a volume of a particular suitable buffer in contact with the solid support is removed so it is replaced with a similar volume of either the same or a different buffer. Thus, buffers applied to the flow cell through an inlet are removed by the outlet by a process of buffer exchange.

Desirably, a means for detecting a signal has sufficient resolution to enable it to distinguish between and among signals generated from different colonies.

Apparatuses of the present invention (of whatever nature) are preferably provided in automated form so that once they are activated, individual process steps can be repeated automatically.

### EXAMPLE 1

### Comparison of Isothermal and Thermal Amplification

Experimental Overview

The following experimental details describe the complete exposition of one embodiment of the invention as described above. Preparation and sequencing of clusters are described in copending patents WO06064199 and WO07010251, whose protocols are included herein by reference in their entirety.

Acrylamide Coating of Glass Chips

The solid supports used are typically 8-channel glass chips such as those provided by Micronit (Twente, Nederland) or IMT (Neuchatel, Switzerland). However, the experimental conditions and procedures are readily applicable to other solid supports such as, for example, Silex Microsystems.

Chips were washed as follows: neat Decon for 30 min, Milli-Q® H₂O for 30 min, NaOH 1N for 15 min, Milli-Q® H₂O for 30 min, HCl 0.1N for 15 min, Milli-Q® H₂O for 30 min.

Polymer Solution Preparation

For 10 ml of 2% polymerisation mix:
10 ml of 2% solution of acrylamide in Milli-Q® H₂O
165 μl of a 100 mg/ml N-(5-bromoacetamidylpentyl)acrylamide (BRAPA) solution in DMF (23.5 mg in 235 μl DMF)
11.5 μl of TEMED
100 μl of a 50 mg/ml solution of potassium persulfate in Milli-Q® H₂O (20 mg in 400 μl H₂O)

The 10 ml solution of acrylamide was first degassed with argon for 15 min. The solutions of BRAPA, TEMED and potassium persulfate were successively added to the acrylamide solution. The mixture was then quickly vortexed and immediately used. Polymerization was then carried out for 1 h 30 at RT. Afterwards the channels were washed with Milli-Q® H₂O for 30 min. The slide was then dried by flushing argon through the inlets and stored under low pressure in a dessicator.

### Synthesis of N-(5-bromoacetamidylpentyl)acrylamide (BRAPA)

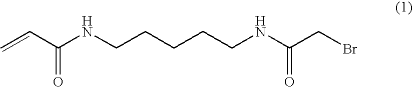

N-Boc-1,5-diaminopentane toluene sulfonic acid was obtained from Novabiochem. The bromoacetyl chloride and acryloyl chloride were obtained from Fluka. All other reagents were Aldrich products. 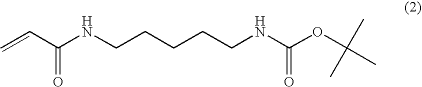
To a stirred suspension of N-Boc-1,5-diaminopentane toluene sulfonic acid (5.2 g, 13.88 mmol) and triethylamine (4.83 ml, 2.5 eq) in THF (120 ml) at 0° C. was added acryloyl chloride (1.13 ml, 1 eq) through a pressure equalized dropping funnel over a one hour period. The reaction mixture was then stirred at room temperature and the progress of the reaction checked by TLC (petroleum ether:ethyl acetate; 1:1). After two hours, the salts formed during the reaction were filtered off and the filtrate evaporated to dryness. The residue was purified by flash chromatography (neat petroleum ether followed by a gradient of ethyl acetate up to 60%) to yield 2.56 g (9.98 mmol, 71%) of product 2 as a beige solid. ¹H NMR (400 MHz, d₆-DMSO): 1.20-1.22 (m, 2H, CH₂), 1.29-1.43 (m, 13H, tBu, 2×CH₂), 2.86 (q, 2H, J=6.8 Hz and 12.9 Hz, CH₂), 3.07 (q, 2H, J=6.8 Hz and 12.9 Hz, CH₂), 5.53 (dd, 1H, J=2.3 Hz and 10.1 Hz, CH), 6.05 (dd, 1H, J=2.3 Hz and 17.2 Hz, CH), 6.20 (dd, 1H, J=10.1 Hz and 17.2 Hz, CH), 6.77 (t, 1H, J=5.3 Hz, NH), 8.04 (bs, 1H, NH). Mass (electrospray+) calculated for C₁₃H₂₄N₂O₃ 256, found 279 (256+Na⁺).

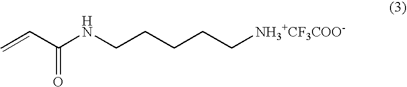

Product 2 (2.56 g, 10 mmol) was dissolved in trifluoroacetic acid:dichloromethane (1:9, 100 ml) and stirred at room temperature. The progress of the reaction was monitored by TLC (dichloromethane:methanol; 9:1). On completion, the reaction mixture was evaporated to dryness, the residue co-evaporated three times with toluene and then purified by flash chromatography (neat dichloromethane followed by a gradient of methanol up to 20%). Product 3 was obtained as a white powder (2.43 g, 9 mmol, 90%). ¹H NMR (400 MHz, D₂O): 1.29-1.40 (m, 2H, CH₂), 1.52 (quint., 2H, J=7.1 Hz, CH₂), 1.61 (quint., 2H, J=7.7 Hz, CH₂), 2.92 (t, 2H, J=7.6 Hz, CH₂), 3.21 (t, 2H, J=6.8 Hz, CH₂), 5.68 (dd, 1H, J=1.5 Hz and 10.1 Hz, CH), 6.10 (dd, 1H, J=1.5 Hz and 17.2 Hz, CH), 6.20 (dd, 1H, J=10.1 Hz and 17.2 Hz, CH). Mass (electrospray+) calculated for C₈H₁₆N₂O 156, found 179 (156+Na⁺).

To a suspension of product 3 (6.12 g, 22.64 mmol) and triethylamine (6.94 ml, 2.2 eq) in THF (120 ml) was added bromoacetyl chloride (2.07 ml, 1.1 eq), through a pressure equalized dropping funnel, over a one hour period and at −60° C. (cardice and isopropanol bath in a Dewar). The reaction mixture was then stirred at room temperature overnight and the completion of the reaction was checked by TLC (dichloromethane:methanol 9:1) the following day. The salts formed during the reaction were filtered off and the reaction mixture evaporated to dryness. The residue was purified by chromatography (neat dichloromethane followed by a gradient of methanol up to 5%). 3.2 g (11.55 mmol, 51%) of the product 1 (BRAPA) were obtained as a white powder. A further recrystallization performed in petroleum ether:ethyl acetate gave 3 g of the product 1. ¹H NMR (400 MHz, d₆-DMSO): 1.21-1.30 (m, 2H, CH₂), 1.34-1.48 (m, 4H, 2×CH₂), 3.02-3.12 (m, 4H, 2×CH₂), 3.81 (s, 2H, CH₂), 5.56 (d, 1H, J=9.85 Hz, CH), 6.07 (d, 1H, J=16.9 Hz, CH), 6.20 (dd, 1H, J=10.1 Hz and 16.9 Hz, CH), 8.07 (bs, 1H, NH), 8.27 (bs, 1H, NH). Mass (electrospray+) calculated for C₁₀H₁₇BrN₂O₂ 276 or 278, found 279 (278+H⁺), 299 (276+Na⁺).

The Cluster Formation Process

Fluidics:

For all fluidic steps during the cluster formation process, a peristaltic pump Ismatec IPC equipped with tubing Ismatec Ref 070534-051 (orange/yellow, 0.51 mm internal diameter) was used. The pump was run in the forward direction (pulling fluids). A waste dish was installed to collect used solution at the outlet of the peristaltic pump tubing. During each step of the process, the different solutions used were dispensed into 8 tube microtube strips, using 1 tube per chip inlet tubing, in order to monitor the correct pumping of the solutions in each channel. The volume required per channel was specified for each step.

The pump was controlled by computer run scripts which prompted the user to change solutions as necessary.

Thermal Control

To enable incubation at a substantially isothermal temperature during the cluster formation process, the chip was mounted on top of an MJ-research thermocycler. The chip sits on top of a custom made copper block, which was attached to the flat heating block of the thermocycler. The chip was covered with a small Perspex block and held in place by adhesive tape.

Grafting of Primers

An acrylamide coated chip was placed onto a modified MJ-Research thermocycler and attached to a peristaltic pump as described above. Grafting mix consisting of 0.5 μM of forward primer and 0.5 μM of a reverse primer in 10 mM phosphate buffer (pH 7.0) was pumped into the channels of the chip at a flow rate of 60 μl/min for 75 s at 20° C. The thermocycler was then heated up to 51.6° C. and the chip was incubated at this temperature for 1 hour. During this time, the grafting mix underwent 18 cycles of pumping: grafting mix was pumped in at 15 μl/min for 20 s, then the solution was pumped back and forth (5 s forward at 15 μl/min, then 5 s backward at 15 μl/min) for 180 s. After 18 cycles of pumping, the chip was washed by pumping in 5×SSC/5 mM EDTA at 15 μl/min for 300 s at 51.6° C.

Template DNA Hybridisation

The DNA templates to be hybridised to the grafted chip were diluted to the required concentration (1 pM template) in 5×SSC/0.1% Tween 20. The hybridization mix was pumped through at 98.5° C., 15 μl/min for 300 sec (75 μl total), an additional pump at 100 μl/min for 10 sec (16.7 μl total) was carried out to flush through bubbles formed by the heating of the hybridisation mix.

The temperature was then held at 98.5° C. for 30 s before being cooled slowly to 40.2° C. in 19.5 minutes with the flow rate static. The flow cell was washed by pumping in 0.3×SSC/0.1% Tween 20 at 15 μl/min for 300 sec (75 μl total) at 40.2° C.

Solid-Phase Amplification

The hybridised template molecules were amplified by a bridging polymerase reaction at a substantially isothermal temperature using the grafted primers and different polymerase enzymes.

The flow cells were pumped with extension pre-buffer (20 mM Tris-HCl, pH 8.8, 10 mM (NH₄)₂SO₄, 2 mM MgSO₄, 0.1% Triton X-100, 2 M Betaine and 1.3% DMSO) at 40.2° C., 15 μl/min for 200 s (50 μl total) and then with extension buffer (pre-buffer with 200 μM dNTPs and 0.025 U/μl DNA polymerase) also at 40.2° C., 60 μl/min for 75 sec (75 μl total). The flow cells were incubated at 40.2° C. for 90 s in extension buffer.

The thermocycler temperature was then set and maintained at 37° C. for the whole isothermal amplification process. For each cycle of isothermal amplification, the DNA on the surface of the flow cell was denatured by pumping 0.1 N NaOH at 60 μl/min for 75 s (75 μl total), and then the flow cell was neutralized using 0.3×SSC/0.1% Tween20 at 60 μl/min for 120 s (120 μl total). The flow cell was washed with extension pre-buffer at 60 μl/min for 75 s (75 μl total) and then extension buffer (enzyme pre-buffer with 200 μM dNTPs and 0.04 U/μl DNA polymerase) was pumped into the flow cell at 60 μl/min for 75 s (75 μl total). The flow cell was incubated with extension buffer for 180 s. The denaturation step was then started by pumping through 0.1 N NaOH for the next cycle. This was repeated for 30 cycles. The flow cell was then washed with 0.3×SSC/0.1% Tween 20 at 37° C., 15 μl/min for 300 s (75 μl total) and ready for the following SYBR Green cluster QC step.

SYBR Green-I Staining

The chip was flushed with 100 mM sodium ascorbate in 0.1 M Tris-HCl buffer pH 8.0 for 5 mins at 15 μl/min/channel, followed by a 1/10000 dilution of SYBR Green-I in 100 mM sodium ascorbate in Tris-HCl buffer pH 8.0 for 5 min at 15 μl/min/channel.

Visualisation

The clusters were visualised using an inverted epi-fluorescence microscope equipped with an EXFO Excite 120 illumination system and a CCD detector (ORCA ER from Hamamatsu). The filters used were the xf22 set from Omega Optical. The exposure power was normalised to 1 millijoule for each exposure to minimise photobleaching of the SYBR green.

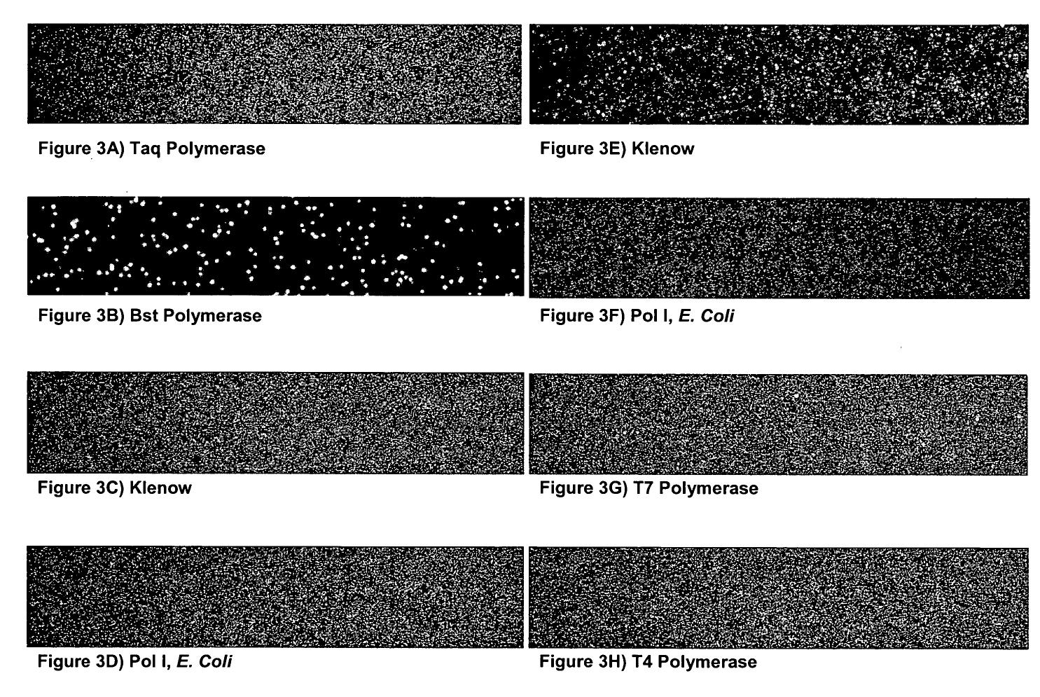

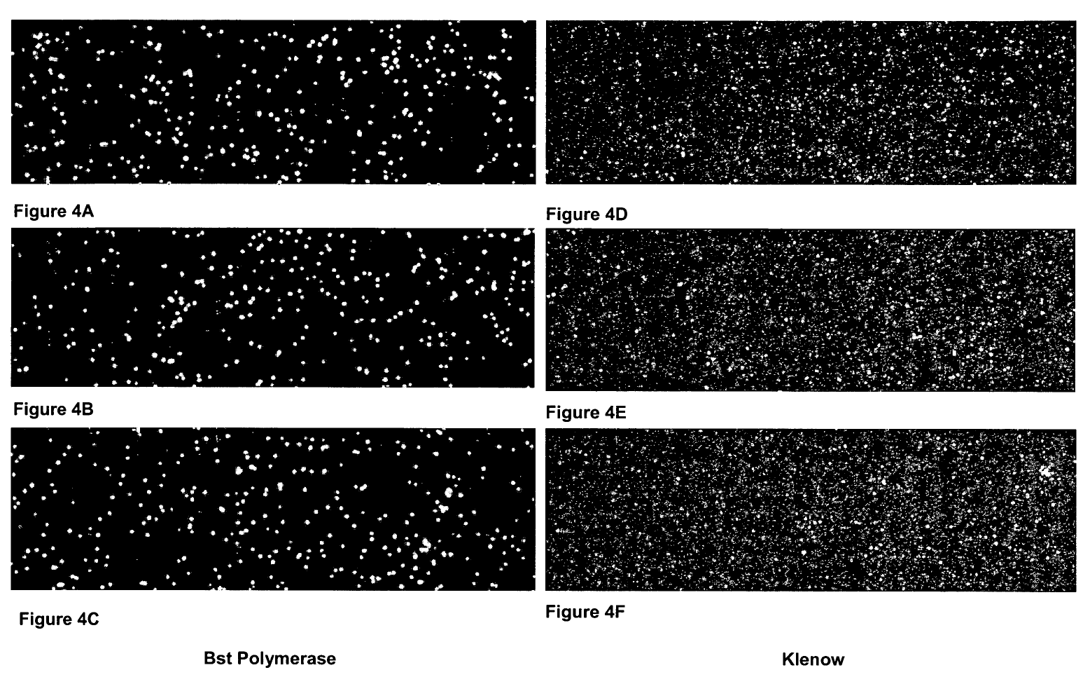

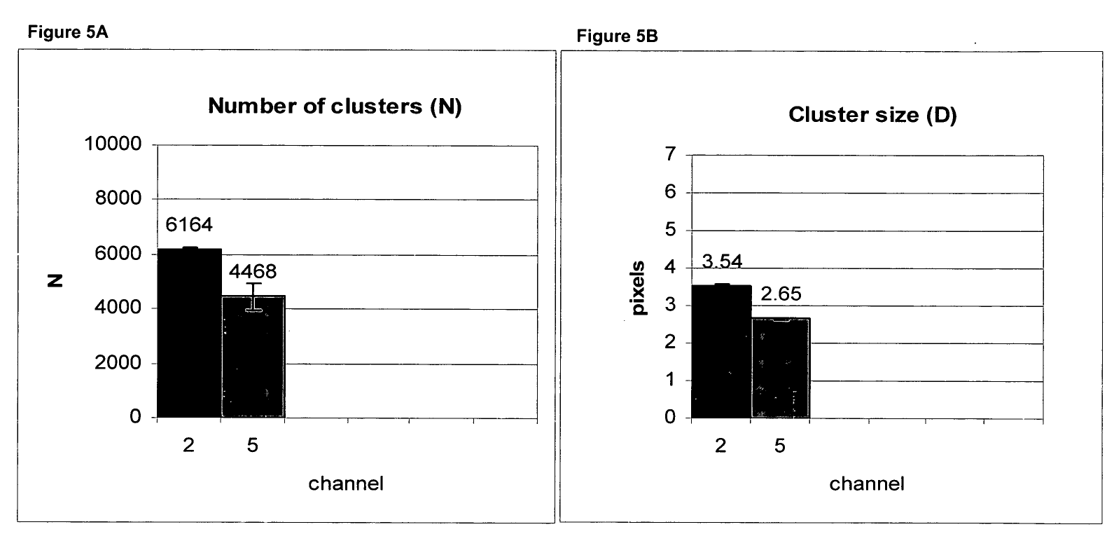

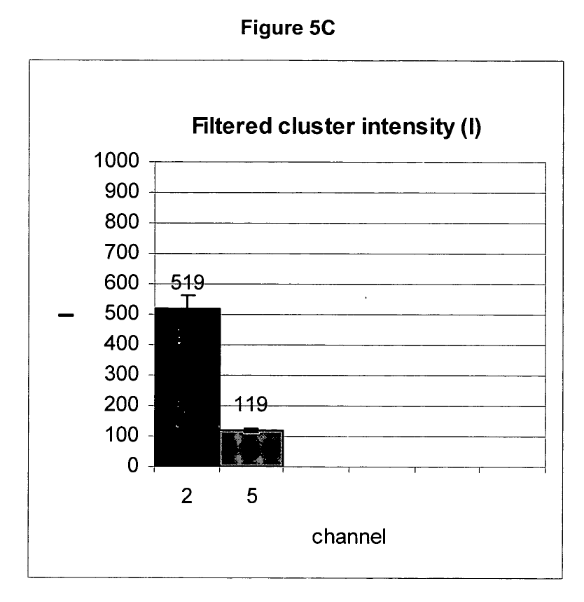

The results of using different DNA polymerase enzymes are shown in FIG. 3. It is apparent that whilst the majority of enzymes gave little signal from the SYBR green stain, the Bst polymerase showed bright signal, revealing a high density of clusters grown from the hybridised templates. FIG. 4 demonstrates clusters isothermally amplified using Bst polymerase or Klenow. FIGS. 5A, 5B and 5C compare characteristics of clusters isothermally amplified using Bst polymerase or Klenow.

Sequencing

The chips grown by isothermal amplification were sequenced alongside chips grown using standard thermocycling methods (as described below). Sequencing results showed no difference in data quality between isothermal and thermocycled clusters, and the correct sequence of the applied template strands could be determined in both cases.

Protocol for Cluster Formation by Thermocycling

1) Template DNA Hybridisation

The DNA templates to be hybridised to the grafted chip are diluted to the required concentration (e.g., 0.5-2 pM) in 5×SSC/0.1% Tween. The diluted DNA is heated on a heating block at 100° C. for 5 min to denature the double stranded DNA into single strands suitable for hybridisation. The DNA is then immediately snap-chilled in an ice/water bath for 3 min. The tubes containing the DNA are briefly spun in a centrifuge to collect any condensation, and then transferred to a pre-chilled 8-tube strip and used immediately.

The grafted chip from step 1 is primed by pumping in 5×SSC/0.1% Tween at 60 μl/min for 75 s at 20° C. The thermocycler is then heated to 98.5° C., and the denatured DNA is pumped in at 15 μl/min for 300 s. An additional pump at 100 μl/min for 10 s is carried out to flush through bubbles formed by the heating of the hybridisation mix. The temperature is then held at 98.5° C. for 30 s, before being cooled slowly to 40.2° C. over 19.5 min. The chip is then washed by pumping in 0.3×SSC/0.1% Tween at 15 μl/min for 300 s at 40.2° C.

2) Amplification Using Thermocycling

The hybridised template molecules are amplified by a bridging polymerase chain reaction using the grafted primers and a thermostable polymerase.

PCR buffer consisting of 10 mM Tris (pH 9.0), 50 mM KCl, 1.5 mM MgCl₂, 1 M betaine and 1.3% DMSO is pumped into the chip at 15 μl/min for 200 s at 40.2° C. Then PCR mix of the above buffer supplemented with 200 μM dNTPs and 25 U/ml Taq polymerase is pumped in at 60 μl/min for 75 s at 40.2° C. The thermocycler is then heated to 74° C. and held at this temperature for 90 s. This step enables extension of the surface bound primers to which the DNA template strands are hybridised. The thermocycler then carries out 50 cycles of amplification by heating to 98.5° C. for 45 s (denaturation of bridged strands), 58° C. for 90 s (annealing of strands to surface primers) and 74° C. for 90 s (primer extension). At the end of each incubation at 98.5° C., fresh PCR mix is pumped into the channels of the chip at 15 μl/min for 10 s. As well as providing fresh reagents for each cycle of the PCR, this step also removes DNA strands and primers which have become detached from the surface and which could lead to contamination between clusters. At the end of thermocycling, the chip is cooled to 20° C. The chip is then washed by pumping in 0.3×SSC/0.1% Tween at 15 μl/min for 300 s at 74° C. The thermocycler is then cooled to 20° C.

### EXAMPLE 2

### Preparation and Sequencing of an Array of Isothermal Clusters Using Formamide Rather than Sodium Hydroxide

Grafting Primers onto Surface of SFA Coated Silex Flowcell

An SFA coated flowcell is placed onto a modified MJ-Research thermocycler and attached to a peristaltic pump. Grafting mix consisting of 0.5 μM of a forward primer and 0.5 μM of a reverse primer in 10 mM phosphate buffer (pH 7.0) is pumped into the channels of the flowcell at a flow rate of 60 μl/min for 75 s at 20° C. The thermocycler is then heated up to 51.6° C., and the flowcell is incubated at this temperature for 1 hour. During this time, the grafting mix undergoes 18 cycles of pumping: grafting mix is pumped in at 15 μl/min for 20 s, then the solution is pumped back and forth (5 s forward at 15 μl/min, then 5 s backward at 15 μl/min) for 180 s. After 18 cycles of pumping, the flowcell is washed by pumping in 5×SSC/5 mM EDTA at 15 μl/min for 300 s at 51.6° C. The thermocycler is then cooled to 20° C.

The primers are typically 5′-phosphorothioate oligonucleotides incorporating any specific sequences or modifications required for cleavage. Their sequences and suppliers vary according to the experiment they are to be used for, and in this case are complementary to the 5′-ends of the template duplex. For the experiment described, the amplified clusters contained a diol linkage in one of the grafted primers. Diol linkages can be introduced by including a suitable linkage into one of the primers used for solid-phase amplification.

The grafted primers contain a sequence of T bases at the 5′-end to act as a spacer group to aid in linearisation and hybridization. Synthesis of the diol phosphoramidite is detailed below. Oligonucleotides were prepared using the diol phosphoramidite using standard coupling conditions on a commercial DNA synthesiser. The final cleavage/deprotection step in ammonia cleaves the acetate groups from the protected diol moiety, so that the oligonucleotide in solution contains the diol modification. The sequences of the two primers grafted to the flowcell are:

5′-TTTTTTTTTTAATGATACGGCGACCACCGA-3′ (SEQ ID NO: 2), wherein a thiophosphate is attached to the 5′ thymidine (T) and a diol moiety is used to link the “T” nucleotide at position 10 to the adenosine (A) nucleotide at position 11;

and

5′-TTTTTTTTTTCAAGCAGAAGACGGCATACGA-3′ (SEQ ID NO; 5), wherein a thiophosphate is attached to the 5′ thymidine (T).

Preparation of diol-phosphoramidite for DNA coupling is described in full in copending patent WO07010251. 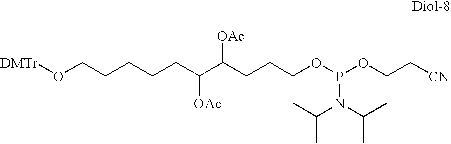
Preparation of Clusters by Isothermal Amplification
Step 1: Hybridisation and Amplification

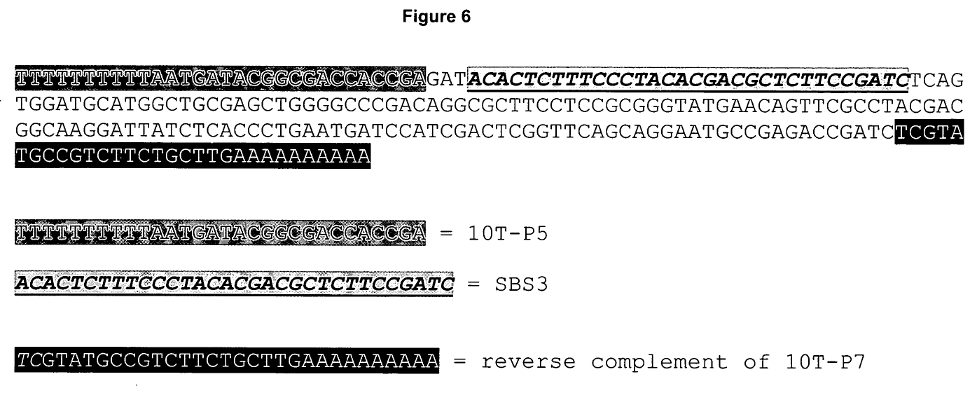

The DNA sequence used in the amplification process is a single monotemplate sequence of 240 bases, with ends complementary to the grafted primers. The full sequence of one strand of the template duplex is shown in FIG. 6. The duplex DNA (1 nM) is denatured using 0.1 M sodium hydroxide treatment followed by snap dilution to the desired 0.2-2 pM ‘working concentration’ in ‘hybridization buffer’ (5×SSC/0.1% Tween).

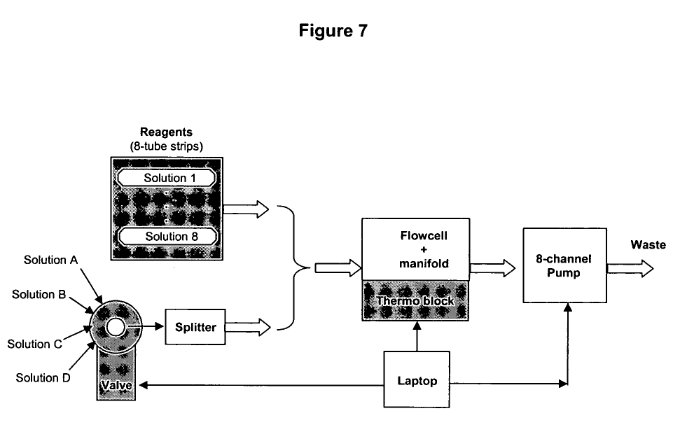

Surface amplification was carried out by isothermal amplification using an MJ Research thermocycler, coupled with an 8-way peristaltic pump Ismatec IPC ISM931 equipped with Ismatec tubing (orange/yellow, 0.51 mm ID). A schematic of the instrument is shown in FIG. 7. To amplify a monotemplate, the same DNA solution is pulled through all 8 channels of the chip.

The single stranded template is hybridised to the grafted primers immediately prior to the amplification reaction, which thus begins with an initial primer extension step rather than template denaturation. The hybridization procedure begins with a heating step in a stringent buffer to ensure complete denaturation prior to hybridisation. After the hybridisation, which occurs during a 20 min slow cooling step, the flowcell was washed for 5 minutes with a wash buffer (0.3×SSC/0.1% Tween).

A typical amplification process is detailed in the following table, detailing the flow volumes per channel:

1. Template Hybridization and 1^{st} Extension

| | | T | Time | Flow rate | Pumped V |
|---|---|---|---|---|---|
| Step | Description | (° C.) | (sec) | (μl/min) | (μl) |
| 1 | Pump Hybridization | 20 | 120 | 60 | 120 |
| | pre-mix | | | | |
| 2 | Pump Hybridization | 98.5 | 300 | 15 | 75 |
| | mix | | | | |
| 3 | Remove bubbles | 98.5 | 10 | 100 | 16.7 |
| 4 | Stop flow and | 98.5 | 30 | static | 0 |
| | hold T | | | | |
| 5 | Slow cooling | 98.5- | 19.5 | static | 0 |
| | | 40.2 | min | | |
| 6 | Pump wash buffer | 40.2 | 300 | 15 | 75 |
| 7 | Pump amplification | 40.2 | 200 | 15 | 50 |
| | pre-mix | | | | |
| 8 | Pump amplification | 40.2 | 75 | 60 | 75 |
| | mix | | | | |
| 9 | First Extension | 74 | 90 | static | 0 |
| 10 | cool to room | 20 | 0 | static | 0 |
| | temperature | | | | |

The instrument is then changed to fit a splitter such that the same reagent solution can be pulled down all the channels of the chip. The splitter is connected to a valve that is used to select which reagents to flow. A four way valve was used to allow selection between the four buffers used in the isothermal amplification process. During amplification, the reagents are flowed across the chip that is held at a constant 60° C.

2. Isothermal Amplification

| | | T | Time | Flow rate | Pumped V |
|---|---|---|---|---|---|
| Step | Description | (° C.) | (sec) | (μl/min) | (μl) |
| (1) | Pump Formamide | 60 | 75 | 60 | 75 |
| This | Pump Amplification | 60 | 75 | 60 | 75 |
| sequence | pre-mix | | | | |
| 35 | Pump Bst mix | 60 | 95 | 60 | 95 |
| times | Stop flow and | 60 | 180 | static | 0 |
| | hold T | | | | |
| 2 | Pump wash buffer | 60 | 120 | 60 | 120 |

Hybridisation pre mix (buffer)=5×SSC/0.1% Tween

Hybridisation mix=0.1 M hydroxide DNA sample, diluted in hybridisation pre mix

Wash buffer=0.3×SSC/0.1% Tween

Amplification pre mix=2 M betaine, 20 mM Tris, 10 mM Ammonium Sulfate, 2 mM Magnesium sulfate, 0.1% Triton, 1.3% DMSO, pH 8.8

Amplification mix=2 M betaine, 20 mM Tris, 10 mM Ammonium Sulfate, 2 mM Magnesium sulfate, 0.1% Triton, 1.3% DMSO, pH 8.8 plus 200 μM dNTP's and 25 units/mL of Taq polymerase (NEB Product ref M0273L)

Bst mix=2 M betaine, 20 mM Tris, 10 mM Ammonium Sulfate, 2 mM Magnesium sulfate, 0.1% Triton, 1.3% DMSO, pH 8.8 plus 200 μM dNTP's and 80 units/mL of Bst polymerase (NEB Product ref M0275L).

Step 2: Linearisation

To linearize the nucleic acid clusters formed within the flow cell channels, the appropriate linearization buffer is flowed through the flow cell for 20 mins at room temp at 15 μL/min (total volume=300 μL per channel), followed by water for 5 mins at room temperature.

The linearisation buffer consists of 1429 μL of water, 64 mg of sodium periodate, 1500 μL of formamide, 60 μL of 1 M Tris pH 8, and 11.4 μL of 3-aminopropanol, mixed for a final volume of 3 mL. The periodate is first mixed with the water while the Tris is mixed with the formamide. The two solutions are then mixed together and the 3-aminopropanol is added to that mixture.

Step 3: Blocking Extendable 3′-OH Groups

To prepare the blocking pre-mix, 1360 μL of water, 170 μL of 10× blocking buffer (NEB buffer 4; product number B7004S), and, 170 μL of cobalt chloride (25 mM) are mixed for a final volume of 1700 μL. To prepare the blocking mix 1065.13 μL of blocking pre-mix, 21.12 μL of 125 μM ddNTP mix, and 3.75 μL of TdT terminal transferase (NEB; part no M0252S) are mixed for a final volume of 1100 μL.

To block the nucleic acid within the clusters formed in the flow cell channels, the blocking buffer is flowed through the flow cell, and the temperature adjusted as shown in the exemplary embodiments below.

| | | T | Time | Flow rate | Pumped V |
|---|---|---|---|---|---|
| Step | Description | (° C.) | (sec) | (μl/min) | (μl) |
| 1 | Pump Blocking | 20 | 200 | 15 | 50 |
| | pre-mix | | | | |
| 2 | Pump Blocking | 37.7 | 300 | 15 | 75 |
| | mix | | | | |
| 3 | Stop flow and | 37.7 | 20 | static | 0 |
| | hold T | | | | |
| 4 | Cyclic pump | 37.7 | 8 × | 15/ | 45 |
| | Blocking mix | | (20 + 180) | static | |
| | and wait | | | | |
| 5 | Pump wash | 20 | 300 | 15 | 75 |
| | buffer | | | | |

Step 4: Denaturation and Hybridization of Sequencing Primer

To prepare the primer mix, 895.5 μL of hybridization pre-mix/buffer and 4.5 μl of sequencing primer (100 μM) are mixed to a final volume of 900 μL. The sequence of the sequencing primer used in this reaction is:

(SEQ ID NO: 3)

5′-ACACTCTTTCCCTACACGACGCTCTTCCGATC-3′.

To denature the nucleic acid within the clusters and to hybridize the sequencing primer, the computer component of the instrumentation flows the appropriate solutions through the flow cell as described below:

| | | T | Time | Flow rate | Pumped V |
|---|---|---|---|---|---|
| Step | Description | (° C.) | (sec) | (μl/min) | (μl) |
| 1 | Pump NaOH | 20 | 300 | 15 | 75 |
| 2 | Pump TE | 20 | 300 | 15 | 75 |
| 3 | Pump Primer | 20 | 300 | 15 | 75 |
| | mix | | | | |
| 4 | Hold at 60 C. | 60 | 900 | 0 | 0 |
| 5 | Pump wash | 40.2 | 300 | 15 | 75 |
| | buffer | | | | |

After denaturation and hybridization of the sequencing primer, the flowcell is ready for sequencing.

DNA Sequencing Cycles were Carried out as Described in National Patent Application Number WO07010251.

Sequencing was carried out using modified nucleotides prepared as described in International patent application WO 2004/018493 and WO2004/018497, and labelled with four different commercially available fluorophores (Molecular Probes Inc.).

A mutant 9°N polymerase enzyme (an exo-variant including the triple mutation L408Y/Y409A/P410V and C223S) was used for the nucleotide incorporation steps.

Incorporation mix, Incorporation buffer (50 mM Tris-HCl pH 8.0, 6 mM MgSO4, 1 mM EDTA, 0.05% (v/v) Tween −20, 50 mM NaCl) plus 110 nM YAV exo-C223S, and 1 μM each of the four labelled modified nucleotides, was applied to the clustered templates, and heated to 45° C.

Templates were maintained at 45° C. for 30 min, cooled to 20° C. and washed with Incorporation buffer, then with 5×SSC/0.05% Tween 20. Templates were then exposed to Imaging buffer (100 mM Tris pH 7.0, 30 mM NaCl, 0.05% Tween 20, 50 mM sodium ascorbate, freshly dissolved).

Templates were scanned in 4 colours at room temperature.

Templates were then exposed to sequencing cycles of Cleavage and Incorporation as follows:

Cleavage

The procedure is as follows:

Prime with Cleavage buffer (0.1 M Tris pH 7.4, 0.1 M NaCl and 0.05% Tween 20). Heat to 60° C.

Treat the clusters with Cleavage mix (100 mM TCEP in Cleavage buffer).

Wait for a total of 15 min in addition to pumping fresh buffer every 4 min.

Cool to 20° C.

Wash with Enzymology buffer.

Wash with 5×SSC/0.05% Tween 20.

Prime with Imaging buffer.

Scan in 4 colours at RT.

Incorporation

The procedure is as follows:

Prime with Incorporation buffer. Heat to 60° C.

Treat with Incorporation mix. Wait for a total of 15 min in addition to pumping fresh Incorporation mix every 4 min.

Cool to 20° C.

Wash with Incorporation buffer.

Wash with 5×SSC/0.05% Tween 20.

Prime with imaging buffer.

Scan in 4 colours at RT.

Repeat the process of Incorporation and Cleavage for as many cycles as required.

Incorporated nucleotides were detected using a total internal reflection based fluorescent CCD imaging apparatus. Images are recorded and analysed to measure the intensities and numbers of the fluorescent objects on the surface. The sequence of the first 25 bases of the sequence extending away from the sequencing primer hybridisation site were successfully determined for the amplified clusters, showing that the isothermal amplification process generates clusters amenable to sequence determination.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for isothermally amplifying single stranded nucleic acid molecules immobilized on a planar solid surface comprising:
i) providing a planar solid surface comprising at least one 5′-end immobilized first single stranded nucleic acid template molecule comprising a sequence Y at the 5′ end and a sequence Z at the 3′ end and a plurality of first and second primers comprising sequences X and Y immobilized at their 5′ ends, wherein sequence X is hybridizable to sequence Z;
ii) annealing said at least one 5′-end immobilized first single stranded nucleic acid template molecule to said first immobilized primers, wherein the first sequence Z of each template molecule is annealed to one of said first immobilized primers comprising sequence X;
iii) performing a primer extension reaction using primer annealed 5′-end immobilized first single stranded nucleic acid template molecules to generate double stranded nucleic acid molecules comprising 5′-end immobilized first and second single stranded nucleic acid molecules, wherein the 5′-end immobilized second single stranded nucleic acid molecules are complementary copies of the 5′-end immobilized first single stranded template nucleic acid molecules and each of the 5′-end immobilized second single stranded nucleic acid molecules comprises a sequence at the 3′ end that is hybridizable to the second primer sequence Y;
iv) flowing a chemical denaturant across the planar solid surface to denature said double stranded nucleic acid molecules to generate 5′-end immobilized first and second single stranded nucleic acid molecules;
v) removing the chemical denaturant and annealing said 5′-end immobilized first and second single stranded nucleic acid molecules to said first and second immobilized primers comprising sequences X and Y;
vi) performing a primer extension reaction using primer annealed 5′-end immobilized first and second single stranded nucleic acid molecules as templates to generate double stranded nucleic acid molecules immobilized at both 5′-ends; and
vii) repeating steps iv) through vi) to generate multiple copies of the nucleic acid molecules on said planar solid surface, wherein steps iv) through vi) are carried out at the same temperature.

2. The method of claim 1, wherein the planar solid surface comprises a plurality of 5′-end immobilized first single stranded nucleic acid template molecules comprising different nucleic acid sequences, wherein amplification of said plurality of 5′-end immobilized first single stranded nucleic acid template molecules produces an array of clusters comprising different sequences.

3. The method of claim 2, wherein said clusters are generated at a density of 10⁴-10⁷ clusters per mm².

4. The method of claim 1, wherein the planar solid surface is a flow cell comprising separate inlets and outlets for buffer exchange.

5. The method according to claim 1, wherein said chemical denaturant is hydroxide.

6. The method according to claim 1, wherein said chemical denaturant is formamide.

7. The method according to claim 1, wherein said chemical denaturant is urea.

8. The method according to claim 1, wherein said chemical denaturant is guanidine.

9. The method according to claim 1, wherein the at least one 5′-end immobilized first single stranded nucleic acid template molecule is generated by extension of an immobilised primer.

10. The method according to claim 1, wherein the at least one 5′-end immobilized first single stranded nucleic acid template molecule and the first and second primers comprise a modification to allow direct immobilisation to the planar solid surface.

11. The method according to claim 1, wherein the immobilisation is by covalent attachment.

12. The method according to claim 11, wherein either of the first or second primers comprises a modification that facilitates detachment of at least a portion of the primer from the surface.

13. The method according to claim 12, comprising an additional step of contacting the multiple copies of the nucleic acid molecules on said planar solid surface with chemicals or enzymes to effectuate release of one or more immobilized first and second single stranded nucleic acid molecules from the planar solid surface.

14. The method according to claim 1, further comprising an additional step of performing at least one sequence determination for one or more of the multiple copies of the nucleic acid molecules on said planar solid surface.

15. The method according to claim 14, wherein the sequence determination is made by incorporating labeled nucleotide(s) or oligonucleotides.

16. The method according to claim 15, wherein the labeled nucleotide(s) or oligonucleotides are incorporated onto one of the immobilized primers.

17. The method as claimed in claim 15, wherein the labeled nucleotide(s) or oligonucleotides are incorporated onto a non-immobilized primer hybridized to one strand of the nucleic acid clusters.

18. A clustered array prepared according to claim 1.


SEQUENCE LISTING (5 sequences)

SEQ ID NO: 1 - DNA, 233 bp, Artificial Sequence
  Synthetic DNA Construct
ttttttttttaatgatacggcgaccaccgagatacactctttccctacacgacgctcttccgatctcagtggatgcatggctgcgagctggggcccgacaggcgcttcctccgcgggtatgaacagttcgcctacgacggcaaggattatctcaccctgaatgatccatcgactcggttcagcaggaatgccgagaccgatctcgtatgccgtcttctgcttgaaaaaaaaaa

SEQ ID NO: 2 - DNA, 30 bp, Artificial Sequence
  Synthetic Oligonucleotide
ttttttttttaatgatacggcgaccaccga

SEQ ID NO: 3 - DNA, 32 bp, Artificial Sequence
  Synthetic Oligonucleotide
acactctttccctacacgacgctcttccgatc

SEQ ID NO: 4 - DNA, 31 bp, Artificial Sequence
  Synthetic Oligonucleotide
tcgtatgccgtcttctgcttgaaaaaaaaaa

SEQ ID NO: 5 - DNA, 31 bp, Artificial Sequence
  Synthetic Oligonucleotide
ttttttttttcaagcagaagacggcatacga